(19) United States
(12) Patent Application Publication
(10) Pub. No.: US 2025/0224405 A1
(43) Pub. Date: Jul. 10, 2025

(54) METHODS FOR IMMUNOAFFINITY ENRICHMENT AND MASS SPECTROMETRY

(71) Applicant: Pierce Biotechnology, Inc., Rockford, IL (US)

(72) Inventors: Bhavinkumar B. Patel, Loves Park, IL (US); John C. Rogers, Rockton, IL (US); Penny J. Jensen, Rockford, IL (US)

(73) Assignee: Pierce Biotechnology, Inc., Rockford, IL (US)

(21) Appl. No.: 19/024,849

(22) Filed: Jan. 16, 2025

Related U.S. Application Data
(63) Continuation of application No. 16/971,487, filed on Aug. 20, 2020.
(60) Provisional application No. 62/636,006, filed on Feb. 27, 2018.

(51) Int. Cl. G01N 33/68

(52) U.S. Cl. CPC G01N 33/6848

(57) ABSTRACT

This disclosure relates to the field of mass spectrometry analysis. In some embodiments, the disclosure relates to methods for detecting and quantifying proteins by enrichment followed by mass spectrometry analysis.

### CROSS-REFERENCE TO RELATED APPLICATIONS

This application is a continuation of co-pending U.S. application Ser. No. 16/971,487, filed Aug. 20, 2020, which is a national phase entry pursuant to 35 U.S.C. § 371 of International Application No. PCT/US2019/019577, filed Feb. 26, 2019, which claims the benefit of priority of U.S. Provisional Application No. 62/636,006, filed Feb. 27, 2018.

### FIELD OF THE INVENTION

This disclosure relates to the field of detection and quantification of proteins, by immunoaffinity enrichment and mass spectrometry.

### BACKGROUND

Mass spectrometry (MS) is increasingly becoming the detection methodology of choice for assaying protein abundance and post-translational modifications. Immunoprecipitation (IP) is commonly used upstream of MS as an enrichment tool for low-abundant protein targets. See, Gingras et al., Nat. Rev. Mol. Cell. Biol., August 2007, 8 (8), 645-54; and Carr, S. A. et al., Mol. Cell. Proteomics March 2014, 13 (3), 907-17. Additional methods of enriching for the protein of interest upstream of MS may also be used. See, e.g., Lin, et al., J. Proteome Res., 2013 Dec. 6; 12 (12); 5996-6003; Schwertman, et al., Analytical Biochemistry, Vol. 440, Issue 2, 15 Sep. 2013, 227-236; and Rafalko, et al., Anal. Chem., 2010, 82 (21), 8998-9005.

The present disclosure provides methods for detecting and quantifying proteins via immunoaffinity enrichment, mass spectrometry (MS), and immunoaffinity enrichment followed by mass spectrometry (IP-MS).

### SUMMARY

In some embodiments, methods are provided for detecting one or more target protein(s) in a biological sample. In some embodiments, methods are provided comprising enriching the target protein(s) from a biological sample by binding the target proteins(s) to a solid support. In embodiments, methods are provided comprising fragmenting enriched target protein(s). In embodiments, methods are provided comprising while bound to the solid support, treating enriched target protein(s) with a first enzymatic digestion, reducing and alkylating the digested target protein(s) concurrently in a single reaction vessel, digesting the reduced, alkylated, and digested target protein(s) in a second enzymatic digestion, wherein the second enzymatic digestion is allowed to proceed for up to 4 to 18 hours. In some embodiments, methods are provided comprising detecting one or more target protein(s) in the sample. In some embodiments, in which enriching the target protein(s) from a biological sample by binding the target protein(s) to a solid support comprises treating the biological sample with at least one antibody capable of immunoaffinity enriching the target protein(s) from a biological sample. In some embodiments, detecting one or more target protein(s) in the sample comprises assaying the fragmented protein(s) via mass spectrometry to determine the presence or absence of at least one peptide from the target protein(s) and detecting one or more target protein(s). In some embodiments, detecting one or more target protein(s) in the sample comprises ELISA, Western blot, Luminex, fluorescence-based imaging, or chemiluminescent-based imaging.

In embodiments, methods are provided in which the reduction and alkylation occurs in a single reaction vessel. In embodiments, methods are provided in which the second enzymatic digestion is allowed to proceed for up to 4 hours. In some embodiments, the peptide from the target protein(s) is less than or equal to 40 amino acids in length.

In some embodiments, methods are provided in which the first and/or second enzymatic digestion comprises digestion with trypsin, chymotrypsin, AspN, GluC, LysC, LysN, ArgC, proteinase K, pepsin, clostripain, elastase, GluC biocarb, LysC/P, LysN promisc, protein endopeptidase, staph protease or thermolysin. In some embodiments, the first and/or second enzymatic digestion comprises digestion with trypsin. In some embodiments, the first and/or second enzymatic digestion comprises digestion with trypsin and LysC. In some embodiments, trypsin is present in the first enzymatic digestion at a concentration of about 0.1 μg/μl to about 0.4 μg/μl. In some embodiments, trypsin is present in the first enzymatic digestion at a concentration of about 0.2 μg/μl. In some embodiments, the trypsin is present in the second enzymatic digestion at a concentration of about 0.02 μg/μl to about 0.08 μg/μl. In some embodiments, the trypsin is present in the second enzymatic digestion at a concentration of about 0.06 μg/μl.

In some embodiments, methods are provided in which the reduction/alkylation step comprises mixing the product of the first enzymatic digestion with a solution comprising TCEP and chloroacetamide. In some embodiments, the TCEP and chloroacetamide are present in a ratio of 1:1, 1:2, 1:3, 1:4, or 1:5. In some embodiments, TCEP is present in a concentration of about 5 to about 10 mM. In some embodiments, chloroacetamide is present in a concentration of about 5 to about 50 mM.

In some embodiments, the methods further comprise the step of neutralization after the second digestion and prior to mass spectrometry. In some embodiments, the neutralization step comprises adding trifluoroacetic acid (TFA) to the product of the second enzymatic digestion.

In some embodiments, methods are provided comprising treating the sample with a labelled antibody capable of binding to the target protein to provide a labelled antibody-protein conjugate; and binding the labelled antibody-protein conjugate with a capture agent capable of binding to the labelled antibody to isolate the target protein from the sample. In some embodiments, the label is biotin and the capture agent is streptavidin.

In some embodiments, the lower limit of detection for the protein(s) is from about 0.04 to about 11.11 fmol.

In some embodiments, methods are provided further comprising determining the quantity of the target protein. In some embodiments, the quantity of a target protein is determined by adding an internal standard peptide of known amount to the digested protein prior to mass spectrometry, wherein the internal standard peptide has the same amino acid sequence as a target peptide, and is detectably labeled, and determining the quantity of a target peptide by comparison to the internal standard. In some embodiments, the quantity of a target protein is determined by a method comprising comparing an amount of a target peptide in the sample to the amount of the same target peptide in a control sample.

In some embodiments, methods are provided further comprising quantifying the relative amount of the target protein. In some embodiments, methods are provided further comprising quantifying the absolute amount of the target protein. In embodiments, the lower limit of quantification is from about 0.04 to about 11.11 fmol.

In some embodiments, methods are provided further comprising desalting after fragmentation and prior to mass spectrometry. In some embodiments, methods are provided further comprising desalting online using C18 trap column prior to liquid chromatography to mass spectrometry analysis.

In some embodiments, the mass spectrometry may be selected from targeted mass spectrometry and discovery mass spectrometry. In some embodiments, the targeted mass spectrometry may be selected from multiple reaction monitoring (MRM), selected reaction monitoring (SRM), and parallel reaction monitoring (PRM), or combinations thereof.

In some embodiments, the biological sample may be selected from isolated cells, plasma, serum, whole blood, CSF, urine, sputum, tissue, and tumorous tissue. In some embodiments, the biological sample is human.

In some embodiments, methods are provided wherein the peptide from the target protein(s) comprises an epitope for the antibody capable of immunoaffinity enriching the target protein(s).

In some embodiments, methods including assessing completion of digestion. A complete digestion comprises 90% or higher zero missed cleavages.

In some embodiments, the method further comprises separating the solid support from digested proteins(s).

### BRIEF DESCRIPTION OF THE FIGURES

FIG. 1 shows a representative improved workflow for an immunoaffinity enriched mass spectrometry assay to identify target proteins.

FIG. 2 shows a comparison of representative workflows for processing immunoaffinity enriched samples.

FIG. 3 shows the results of MS sample prep method for low pH/organic IP elutions compared with a urea-based method.

FIG. 4 shows the results of various conditions for IP elution using enzyme and sequential reduction/alkylation.

FIG. 5 shows IgG levels obtained with enzyme elution compared to IP-MS elution buffer methods.

FIG. 6 shows the recovery of target peptides (as percent of control) obtained with different processing methods. A urea method is used as a control.

FIG. 7 shows recovery of target peptides obtained with urea vs. enzyme elution methods.

FIG. 8 compares recovery of peptides using urea-based method, trypsin elution, and trypsin elution with single pot reduction/alkylation.

FIGS. 9A-9B show results for average top peptide area for different enzyme digestion times. An overnight (O/N) digestion was used as a control and the data are shown as a % of control.

FIGS. 10A-10B show results for average peptide area obtained with different enzyme digestion times and digestion temperatures. An overnight (O/N) digestion was used as a control and the data are shown as a % of control.

FIGS. 11A-11C shows % CV (coefficient of variation) of peptide area obtained from three different experiments in which different enzyme digestion times were compared.

FIGS. 12A-12F show the results of targeted MS analysis of unique peptides for each target across different digestion times presented as % of overnight digestion (control). A) mTOR; B) RAS; C) STAT3; D) RPTOR; E) CTNNB1; F) IQGAP1.

FIGS. 13A-13B show the results of A) peptide intensities and B) % 0 missed cleavage for peptides for different amounts of enzyme(s) in digestions and digestion times. An overnight digestion with 200 ng trypsin was used as a control and the data are shown as a % of control. (T: Trypsin; T+L: Trypsin+LysC)

FIGS. 14A-14F show the results of targeted analysis for unique peptides for each target under different digestion conditions with respect to enzyme, enzyme amount, and time of digestion as compared to an overnight control digestion. A) mTOR; B) RAS; C) STAT3; D) RPTOR; E) CTNNB1; F) IQGAP1.

FIG. 15 provides a flowchart outlining an experimental protocol to test conditions for enzymatic elution of immunoprecipitated material from beads with varying amounts of enzyme and time of initial digestion.

FIGS. 16A-16B show the peptides recovered under different conditions of enzyme IP elution from beads. Graphs are plotted as % Control (trypsin elution (E) using 1 ug of trypsin for 1 hour).

FIGS. 17A-17F show Parallel Reaction Monitoring (PRM) analysis of peptides under different enzyme elution conditions.

FIG. 18 shows a flowchart for an experimental protocol to test conditions for the enzymatic elution of immunoprecipitated material from beads. The grade of trypsin, amount of trypsin, and time of elution digestion are varied.

FIGS. 19A-19B show the results of an experiment to optimize the enzymatic elution of immunoprecipitated material from beads. The grade of trypsin, amount of trypsin, and time of elution digestion were varied. (T: trypsin)

FIGS. 20A-20B show the % CV from two experiments to test conditions for the enzymatic elution of immunoprecipitated material using trypsin.

FIG. 21 summarizes the results from two experiments to test conditions for the enzymatic elution of immunoprecipitated material using 1 μg trypsin.

### DETAILED DESCRIPTION

### I. Definitions

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

As used herein “protein”, “peptide”, and “polypeptide” are used interchangeably throughout to mean a chain of amino acids wherein each amino acid is connected to the next by a peptide bond. In some embodiments, when a chain of amino acids consists of about two to forty amino acids, the term “peptide” is used. However, the term “peptide” should not be considered limiting unless expressly indicated.

The term “antibody” is used in the broadest sense and encompasses various antibody structures, including but not limited to monoclonal antibodies, polyclonal antibodies, multispecific antibodies (such as bispecific antibodies), and antibody fragments so long as they exhibit the desired immunoprecipitating activity. As such, the term antibody includes, but is not limited to, fragments that are capable of binding to an antigen, such as Fv, single-chain Fv (scFv), Fab, Fab′, di-scFv, sdAb (single domain antibody) and (Fab′) 2 (including a chemically linked F(ab′)₂). Papain digestion of antibodies produces two identical antigen-binding fragments, called “Fab” fragments, each with a single antigen-binding site, and a residual “Fc” fragment. Pepsin treatment yields a F(ab′)₂ fragment that has two antigen-binding sites. The term antibody also includes, but is not limited to, chimeric antibodies, humanized antibodies, and antibodies of various species such as mouse, goat, horse, sheep, chicken, etc. Furthermore, for all antibody constructs provided herein, variants having the sequences from other organisms are also contemplated, such as CDR-grafted antibodies or chimeric antibodies. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct. The antibodies are referred to by reference to name and catalog reference. The skilled artisan, holding this name and catalog information, is capable of determining the sequence of the antibody, and therefore this application encompasses any antibody having at least partial sequence of a reference antibody so long as the antibody maintains its ability to immunoaffinity enrich its antigen protein.

“Immunoaffinity enrichment” refers to any antibody-driven enrichment step. It includes, but is not limited to, methods in which a precipitate is formed, such as “immunoprecipitation.”

Mass spectrometry (MS) is a technique for analysis of proteins on the basis of their mass-to-charge ratio (m/z). MS techniques generally include ionization of compounds and optional fragmentation of the resulting ions, as well as detection and analysis of the m/z of the ions and/or fragment ions followed by calculation of corresponding ionic masses. A “mass spectrometer” generally includes an ionizer and an ion detector. “Mass spectrometry,” “mass spec,” “mass spectroscopy,” and “MS” are used interchangeably throughout.

“Targeted mass spectrometry,” also referred to herein as “targeted mass spec,” “targeted MS,” and “tMS” increases the speed, sensitivity, and quantitative precision of mass spec analysis. Non-targeted mass spectrometry, sometimes referred to as “data-dependent scanning,” “discovery MS,” and “dMS” and targeted mass spec are alike in that in each, analytes (proteins, small molecules, or peptides) are infused or eluted from a reversed phase column attached to a liquid chromatography instrument and converted to gas phase ions by electrospray ionization. Analytes are fragmented in the mass spec (a process known as tandem MS or MS/MS), and fragment and parent masses are used to establish the identity of the analyte. Discovery MS analyzes the entire content of the MS/MS fragmentation spectrum. In contrast, in targeted mass spectrometry, a reference spectrum is used to guide analysis to only a few selected fragment ions rather than the entire content.

“Multiple reaction monitoring,” “MRM,” “selected reaction monitoring,” and “SRM” are used interchangeably throughout to refer to a type of targeted mass spectrometry that relies on a unique scanning mode accessible on triple-quadrupole (QQQ) instruments. See, e.g., Chambers et al., Expert Rev. Proteomics, 1-12 (2014).

“Parallel Reaction Monitoring,” and “PRM” are used interchangeably herein to describe another type of targeted mass spec wherein the second mass analyzer used in SRM (quadrupole) is substituted by a high resolution orbitrap mass analyzer in PRM. Unlike SRM, which allows the measuring of one single transition at a given point in time, PRM allows parallel monitoring in one MS/MS spectrum. PRM also allows for the separation of ions with close m/z values (i.e., within a 10 ppm range), and may therefore allow for lower limits of detection and quantification (LOD or LLOD and LOQ or LLOQ).

To assess completion of digestion of the target protein, the number of “missed cleavages” is calculated. For example, the enzyme trypsin cuts the protein at the C-terminal side of lysine (K) or arginine (R) residues. If a peptide has a single internal K or R as well as the C-terminal K or R, that peptide has one missed cleavage. If a peptide only has the C-terminal K or R, that peptide has zero missed cleavage. If a peptide has a total of two internal K or R residues as well as the C-terminal K or R, that peptide has two missed cleaves. The same holds true for other enzymes and the residues they cleave at.

In some embodiments, a complete digestion can comprise 90%, 91%, 92%, 93%, 94%, 95%, 96%, 97%, 98%, 99%, or 100% zero missed cleavages. In some embodiments, a complete digestion may comprise 1%, 2%, 3%, 4%, or 5% two missed cleavages. In some embodiments, a complete digestion may comprise 1%, 2%, 3%, 4%, 5%, 6%, 7%, 8%, 9% or 10% one missed cleavages.

### II. Improved Methods of Sample Preparation for Immunoaffinity Enrichment/Mass Spectrometry Methods

Improved methods for sample preparation for immunoaffinity enrichment/mass spectrometry methods include those that have the benefit of a shorter digestion time in a second enzymatic digestion, allowing for an improved user workflow and less time from initial immunoaffinity enrichment through mass spectrometry. Thus, a method for detecting one or more target protein(s) in a biological sample, comprises:
a. enriching the target protein(s) from a biological sample by binding the target protein(s) to a solid support;
b. fragmenting the enriched target protein(s) by:
   i. while bound to the solid support, treating the enriched target protein(s) with a first enzymatic digestion,
   ii. reducing and alkylating the digested target protein(s) in a single reaction vessel, and
   iii. digesting the reduced, alkylated, and digested target protein(s) in a second enzymatic digestion, wherein the second enzymatic digestion is allowed to proceed for up to 4 to 18 hours;
c. detecting one or more target protein(s) in the sample.

In some embodiments, target proteins are bound to a solid support comprising a bead or a resin. In some embodiments, target proteins are bound to a solid support comprising a magnetic bead. In some embodiments, target proteins are bound to a solid support comprising an immunoaffinity bead.

In embodiments, a sample that has been enriched (including immunoaffinity enrichment) for one or more target proteins may be subjected to an elution step to separate the antibody-protein complex from a solid support. In embodiments, the elution may be an enzymatic elution. In embodiments, an elution may be performed with a low pH/organic reagent. In embodiments, an enriched sample (including an immunoaffinity-enriched sample) may first be subjected to an enzymatic elution, and remaining antibody-protein complex bound to a substrate may subsequently be subjected to a low pH/organic elution.

In embodiments, the present disclosure provides methods of processing enriched biological samples (including immunoaffinity-enriched samples) for MS analysis. In embodiments, the samples are low amount samples (<10 microgram). In embodiments, methods described herein may be used to determine antibody epitope, specificity, and/or antigen in protein complexes.

### A. Immunoaffinity Enrichment

In some embodiments, methods of immunoaffinity enriching a target protein are provided, comprising contacting a biological sample with at least one antibody. The immunoaffinity enriching method may be single-plex or multi-plex. A “single-plex” method utilizes one antibody per assay, whereas a “multi-plex” method utilizes more than one antibody per assay. Immunoaffinity enrichment may or may not comprise immunoprecipitation.

### B. Reduction and Alkylation

In embodiments, the enriched protein(s) (including immunoaffinity-enriched samples) are subjected to reduction and alkylation. The enriched target proteins may be reduced and alkylated prior to fragmentation (e.g., digestion). Samples that have been reduced and alkylated may comprise modifications, such as cysteine residues. In embodiments, reducing and alkylating may take place sequentially. In embodiments, reducing and alkylating may take place in a single reaction vessel.

### C. Digestion

The present method comprises two digestion steps used to fragment the enriched target protein(s) (including immunoaffinity-enriched samples): a first after enriching the target protein(s) and a second after reducing and alkylating the target proteins(s). In some embodiments, protein samples are denatured or solubilized before fragmentation.

In embodiments, the digestion is enzymatic. In embodiments, enzymatic digestion includes, but is not limited to, digestion with a protease such as, for example, trypsin, chymotrypsin, AspN, GluC, LysC, LysN, ArgC, GluC, proteinase K, pepsin, Clostripain, Elastase, LysC/P, LysN Promisc, Protein Endopeptidase, Staph Protease or thermolysin. In some embodiments, the fragmentation protocol uses MS-grade commercially available proteases. In some embodiments, a mixture of different proteases is used (for example, trypsin and LysC). In some embodiments, the digestion is incomplete in order to see larger, overlapping peptides. In some embodiments, the antibody digestion is performed with IdeS, IdeZ, pepsin, or papain to generate large antibody domains for “middle-down” protein characterization. In some embodiments, the fragmentation protocol uses trypsin that is modified.

In some embodiments, the first digestion step is for about 5 minutes to about 4 hours, from about 10 minutes to about 1.5 hours, from about 15 minutes to about 1 hour. In some embodiments, the first digestion step is about 15 minutes, about 30 minutes, or about 1 hour, or up to about 15 minutes, up to about 30 minutes, or up to about 1 hour.

In some embodiments, the second digestion (i.e., of the reduced and alkylated target protein(s)) may proceed for about up to 4 hours, up to 3 hours, up to 2 hours, or up to 1 hour. In some embodiments, the second digestion step may proceed for about 4 hours, about 3 hours, about 2 hours, or about 1 hour. In some embodiments, the second digestion step may proceed from about 1 hour to about 4 hours.

In some embodiments, a step is included to end the digestion step. The step to end the digestion protocol may be addition of a stop solution or a step of spinning or pelleting of a sample. In some embodiments, the digestion is followed by guanidination.

In some embodiments, the fragmentation protocol is carried out in solution. An exemplary commercially available kit for performing in-solution digestion is the In-Solution Tryptic Digestion and Guanidination Kit (Thermo Fisher Cat #89895).

In some embodiments, the fragmentation protocol uses beads. In some embodiments, the fragmentation protocol comprises on-bead digestion. In some embodiments, agarose beads or Protein G beads are used. In some embodiments, magnetic beads are used.

In some embodiments, the completion of digestion is assessed by calculating the number of zero missed cleavage peptides after MS analysis or the number of zero, one, and/or two missed cleavage peptides.

### D. Mass Spectrometry

The methods disclosed herein may be applied to any type of MS analysis. The disclosure is not limited by the specific equipment or analysis used. The use of any equipment with the intent of analyzing the m/z of a sample would be included in the definition of mass spectrometry. Non-limiting examples of MS analysis and/or equipment that may be used include electrospray ionization, ion mobility, time-of-flight, tandem, ion trap, MRM, SRM, MRM/SRM, PRM, and Orbitrap. The disclosure is neither limited by the type of ionizer or detector used in the MS analysis nor by the specific configuration of the MS. The disclosure is not limited to use with specific equipment or software. The disclosure is not limited to the equipment and software described in the Examples.

In embodiments, the samples may optionally be desalted prior to analysis by mass spectrometry.

In some embodiments, after fragmentation (e.g., digestion), peptide samples are analyzed by mass spectrometry (MS), and the resulting spectra are compared with theoretical spectra from known proteins to determine the peptides and proteins in a sample.

Typically, targeted MS is performed by quantifying specific unique peptides of the protein. In some embodiments, known amounts of isotope-labeled (e.g., heavy isotope-labeled) versions of these targeted peptides can be used as internal standards for absolute quantitation. In some instances, proteins of interest are not detectable even after identifying unique peptide standards. The combination of specific antibodies with specific target peptides has allowed the inventors to improve the sensitivity of detection target proteins by MS and has allowed for lower levels of detection and lower levels of quantification than previously seen.

In some embodiments, protein samples are separated using liquid chromatography before MS analysis. In some embodiments, fragmented samples are separated using liquid chromatography before MS analysis.

In some embodiments, peptides used in the MS methods described herein have limits of detection considered to be useful in clinical and research methods. In some embodiments, the peptides are detectably labelled.

In embodiments, kits are provided comprising reagents for performing methods described herein.

### EXAMPLES

The following examples are provided to illustrate certain disclosed embodiments and are not to be construed as limiting the scope of this disclosure in any way.

### Example 1—Low pH/Organic IP Elutions Compared with Urea-Based Method

Experiments were performed to evaluate different MS sample prep methods for low pH/organic IP elutions compared with a urea-based (control) method. Various sample preparation methods were evaluated to decrease time/hands on/speed vac time by performing IP-MS using multiple antibodies mixture. The following two parameters were tested: 1) IP elution with MS compatible buffer and sequential reduction/alkylation including, as described in more details below, a) Control: Urea method; b) Adjust IP with 1M TEAB (No Urea); c) 50 mM TEAB; d) 50 mM TEAB/30% Acetonitrile and 2) IP elution with MS compatible buffer and one pot reduction/alkylation including a) Spin-column device with SDS; b) Spin-column device without SDS; c) PreOmics in-solution digestion kit. The following materials were used for this experiment as described below in Table 1.

TABLE 1
MATERIAL:

| | | Catalog/ |
|---|---|---|
| Material | Vendor | Product No. |
| Streptavidin Magnetic Beads | Thermo Fisher Scientific | 88817 |
| Pierce IP Lysis Buffer | Thermo Fisher Scientific | 87788 |
| Wash buffer A, Wash buffer | Thermo Fisher Scientific | 90408 |
| B and Elution buffer | | |
| 1M triethylammonium | Thermo Fisher Scientific | 90114 |
| bicarbonate (TEAB) | | |
| Urea | Thermo Fisher Scientific | 29700 |
| 0.5M Tris[2-Carboxy- | Thermo Fisher Scientific | 77720 |
| ethyl]phosphine (TCEP) | | |
| Recombinant GFP | Thermo Fisher Scientific | 88899 |
| 20 μg Trypsin | Thermo Fisher Scientific | 1862748 |
| Iodoacetamide (IAA) | Thermo Fisher Scientific | 90034 |

TABLE 2

| Target | Antibody | Amount per IP (μg) |
|---|---|---|
| mTOR | PA1-188 | 3 |
| STAT3 | 13-7000 | 1 |
| PAK1 | 71-9300 | 1 |
| Ras | MA1-012 | 2 |
| IQGAP1 | 33-8900 | 1 |
| CTNNB1 | 71-2700 | 1 |
| RPTOR | 42-4000 | 1 |

### Experiment Protocol:

Step 1. Prepared biotinylated antibody mixture as shown in Table 2 above and mixed with 0.5 mg each of HCT116 (IGF Stim: Unstim)/HEK293 lysates. Performed total of 25 IP reactions by adding appropriate amount of antibody mix (97.9 μL/5 mL Lysate) in 5 mL low protein binding tube. Parafilmed each tube and incubated overnight at 4° C. mixing on a rotator. After incubation added streptavidin magnetic beads using ratio of 1:5 (Antibody: Beads, Since 10 μg Antibody was used 50 μL beads were added per 1 mL). Washed beads with 2× volume cold IP Lysis Buffer twice. Removed wash buffer then added back original volume of IP Lysis Buffer to each tube. Pooled all IPs together in a 50 mL conical tube and aliquoted into 25 1 mL tubes and then added washed beads to each IP. Mixed antibody/antigen samples and rotated for 1 hour at room temperature. Washed 3 times with 500 μL Wash Buffer A. Washed 2 times with 500 μL Wash Buffer B.

Step 2. The following MS sample prep solutions were prepared: 50 mM TEAB—Diluted 1M Trimethylammonium Bicarbonate (TEAB) (PN #90114) to 50 mM by adding 0.5 mL TEAB in 9.5 mL MS grade water, pH 8.5; Denaturation Solution (6M Urea, recombinant GFP)—Added 360 mgs Urea to 675 μL 50 mM TEAB and vortex (exothermic reaction). Added 400 μL of this solution to the GFP standard tube 50 ng; 50 mM TEAB in 30% ACN-Added 500 μL 100 mM TEAB to 300 μL ACN and 200 μL MS grade water; 10 mM TCEP—Diluted 10 μL 0.5M TCEP (PN #77720) with 490 μL 50 mM TEAB, pH8.5; 5 mM TCEP—Diluted 5 μL 0.5M TCEP with 495 μL 50 mM TEAB, pH8.5; 0.5 mM Iodoacetamide (IAA)—Dissolved 9.3 mgs No-weigh IAA (PN #90034) in 100 μL MS-Grade water (Protect from light); 0.1 μg/μL Trypsin Stock—Dissolved 20 μg Trypsin protease (PN #90057) in 200 μL 0.1% Acetic acid, stored as 30 μL aliquots at −80° C.

Step 3. Mass Spec Sample Prep Various Methods tested included. Samples 1-3=6M Urea; samples 4-6=No Urea—50 mM TEAB+30% ACN; samples 7-9=Preomics Kit; samples 10-12=LysC Elution from Bead; samples 13-15=Trypsin Elution from Bead; samples 16-18=LysC+Trypsin Elution from Bead; samples 19-21=On bead Digestion—Remove from bead then Trypsin; samples 22-25=On bead Digestion overnight—Kept on bead then Trypsin. The following elution methods were tested. Samples 1-9 were eluted with 220 μL IP-MS Elution buffer (0.5% formic acid; 30% acetonitrile solution) Vortexed well, quick spin and let sit for 10 mins at room temperature, vortexed and quick spin and put on magnet then removed as much as possible into 2, 2 mL tubes to pool the elutes together. Spun pooled lysates at 15000×g for 2 mins and put on magnet then remove eluted and divided into 205 μL per tube into 10 different tubes labeled 1-9. Dried the samples in speed vac >1 hr (35° C.)—checking every 30 mins vortexing and drying until done; did not see any pellet. Samples 10-12 were eluted by adding 1 μg LysC (PN #90051) in 100 μL 50 mM TEAB to each sample. This solution was made by adding 5 μL of 0.2 μg/μl solution+95 μl 50 mM TEAB per sample. Samples 13-15 were eluted by adding 1 μg Trypsin (PN #1862748) in 100 μL 50 mM TEAB. This solution was made by adding 5 μL of 0.2 μg/μl solution+95 μl 50 mM TEAB per sample. Samples 16-18 were eluted by adding 1 μg LysC/Trypsin in 100 μL 50 mM TEAB. This solution was made by adding 5 μL of 0.2 μg/μl LysC/Trypsin solution+95 μl 50 mM TEAB per sample. All samples 10-18 were followed by incubation at 37° C. 800 rpm for 1.5 hour. Then beads were collected on magnet and 90 μl of supernatant was removed to a clean tube. This was followed by addition of 2 μl 25 ng/μl recombinant GFP and 0.92 μl 0.5M TCEP and then incubated at 60° C. for 30 mins. For Samples 19-24, prepared 60 μl of Trypsin plus 540 μl 50 mM TEAB and added 100 μl of this solution to each and incubated at 37° C. at 800 rpm for 1 hour. After 1 hour of incubation 1 μl 0.5M TCEP and 2 μl 25 ng/μl GFP were added and incubated for 30 mins at 37° C. at 800 rpm.

Step 4. The sample prep continued for trypsin digestion as described below: For Samples 1-3—Suspended the dried sample in 10 μL 6M Urea/TEAB/GFP solution and vortexed for 30 seconds followed by addition of 10 μL 10 mM TCEP mixed and incubated at 35° C. mixing at 1000 rpm for 30 mins; For Samples 4-6-Resuspended in 20 μL of solution containing 50 mM TEAB/30% ACN, 50 ng of rGFP. Incubated at 60° C. for 30 mins at 500 rpm. For Samples 7-9—Kept dried down pellet at −20° C. for Preomics kit the next day.

Step 5. Performed alkylation of peptides using IAA as described below: For Samples 1-6-Added 1 μL IAA solution mix; For Samples 10-18-Added 4.6 μL IAA; For Samples 19-24: Added 5.15 μL IAA. Incubate all 30 mins RT protected from light.

Step 6. Performed trypsin digestion as described below: For Samples 1-6-Immediately added 45 μL 50 mM TEAB, pH 8.5. Prepared 20 ng/μL trypsin solution by adding 120 μL 50 mM TEAB solution to 30 μL aliquots of 0.1 μg/μL Trypsin Stock and then added 10 μL of this to samples 1-18. Volume at this point was 76 μL (Samples 1-6), 107.676 μL (Samples 10-18). For samples 19-21, removed 92 μL from beads then added the 10 μL of trypsin. For samples 22-24, kept on bead for trypsin digestion. Incubated all samples at 37° C. for 18.5 hrs at 500 rpm.

Step 7. Prepared a mixture of 0.2% Formic Acid (FA), 4% Acetonitrile (ACN) to resuspend the dried samples by adding 940 μL MS grade water, 20 μL 10% FA and 40 μL of 100% ACN. Removed 92 μL of samples 22-24 from the beads. Acidified the samples by adding 3.5 μL 10% TFA (pH <3). Added 4.5 μL of 10% TFA to acidify samples 10-24. Centrifuged all samples at 15,000×g for 2 mins. Removed 65 μL of samples 1-6, 90 μL of samples 10-18 and 82 μL of Samples 19-24 to a new low protein binding tube. Dried down the samples for about 1 hr. Added 0.2% FA and 4% ACN to each tube as described below: 13 μL for samples 1-3 and 17 μL for all remaining samples and vortexed to mix. Stored all samples at −20° C. before nanoLC-MS/MS analysis.

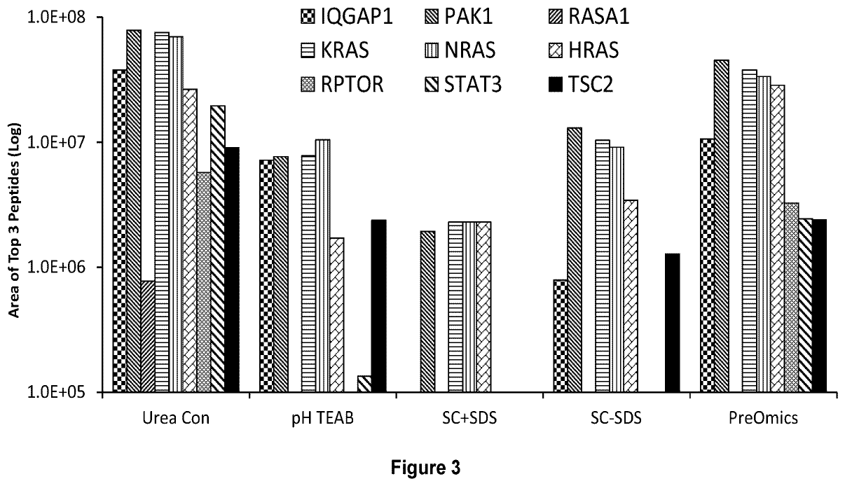

As shown in FIG. 3, the urea method worked best compared to no urea and device-assisted methods.

### Example 2—Enzyme Elutions Compared with Low pH/Organic IP Elution Method

Another experiment was performed to evaluate various sample prep methods by performing IP-MS using QC Mix Antibodies. Sequential reduction and alkylation (with/without beads) was followed by a second trypsin digestion overnight.

Step 1. Prepared QC Mix for 15 IPs as shown in Table 2.

Step 2. Used 0.5 mg each of HCT116 (IGF Stim: Unstim)/HEK293 lysates per IP. Set up 15 IP reactions by adding appropriate amount of antibody mix to 1 mg of lysate per IP. Used parafilm to cover the tube caps and rotated overnight on 4° C.

Step 3. Used 50 μL beads for each IP using ratio of 1:5 for antibody amount to bead volume. Washed beads with 2× volume cold IP Lysis Buffer twice. Added 1 mL of antigen-antibody mixture to 50 μL beads and rotated for 1 hr at room temperature. Washed 3 times with 500 μL of Wash Buffer A followed by 2 times with 500 μL of Wash Buffer B. Evaluated the following methods for Mass Spec Sample Prep: samples 1-3=elution buffer followed by Urea in-solution digestion; samples 4-6=Trypsin Elution from Bead; samples 7-9=LysC+Trypsin Elution from Bead; samples 10-12=On bead Digestion overnight-Keep on bead then Trypsin; samples 13-15=On bead Digestion-removed from bead then Trypsin.

Step 4. Eluted Samples 1-3 with 220 μL IP-MS Elution buffer for 10 mins at room temperature. Placed on magnet and removed 220 μL to a new 1.5 mL low-protein binding tubes to pool the elutes together. Centrifuged the pooled samples at 15000×g for 2 mins and put on magnet then pull elute divide into 205 μL per tube into 3 different tubes labeled 1-3. Dried the samples in speed vac >1 hr (35° C.). Eluted samples 4-6 by adding 1 μg Trypsin in 100 μL 50 mM TEAB and incubated at 37° C. 800 rpm for 1.5 hours. After incubation, put on magnet to remove beads and took 90 μl supernatant followed by addition of 2 μl 25 ng/μl GFP and 0.92 μl 0.5M TCEP and incubate 60° C. for 30 minutes. Eluted samples 7-9 by adding 1 μg LysC/Trypsin in 100 μL 50 mM TEAB, pH 8.5 and incubated at 37° C. 800 rpm for 1.5 hours. Eluted samples 10-15 by adding 1 μg of Trypsin in 50 mM TEAB buffer and incubated at 37° C. at 800 rpm for 1 hour. After 1 hr of incubation, added 1 μl 0.5M TCEP and 2 μl 25 ng/μl rGFP and incubated for 30 mins at 37° C. at 800 rpm.

Step 5. Prepared the following solution for the next step of sample prep: Denaturation Solution—6M Urea+GFP-Used 360 mgs aliquots of Urea by adding 675 μL 50 mM TEAB and vortex (exothermic reaction). Added 400 μL of this solution to the GFP standard tubes 50 ng; 10 mM TCEP—Diluted 10 μL 0.5M TCEP with 490 μL 50 mM TEAB, pH8.5; 5 mM TCEP—Diluted 5 μL 0.5M TCEP with 495 μL 50 mM TEAB, pH8.5; 0.5 mM IAA—Dissolved No-weigh IAA 9.3 mgs in 100 μL MS-Grade water (Protect from light); 0.1 μg/μL Trypsin Stock—Dissolved 20 μg Trypsin protease (P #90057) in 200 μL 0.1% Acetic acid, stored as 30 μL aliquots at −80° C. 20 ng/μL trypsin working solution (prepared just before use)—Added 120 μL 50 mM TEAB solution to 30 μL aliquots of 0.1 μg/μL trypsin stock; 0.2% Formic Acid (FA), 4% Acetonitrile (ACN)—Added 940 μL MS grade water to 20 μL 10% FA and 40 μL of 100% ACN.

Step 6. For samples 1-3, added 10 μL 6M Urea/50 mM TEAB/rGFP solution to dried samples and vortexed for 30 seconds. Added 10 μL 10 mM TCEP mix and incubate at 35° C. mixing at 1000 rpm for 30 mins. Added 1 μL IAA for samples 1-3, 4.6 μL IAA for samples 4-9, 5.15 μL IAA for samples 10-15. Incubated all samples for 30 minutes at room temperature protected from light. Added 45 μL 50 mM TEAB, pH 8.5 to samples 1-3. For samples 13-15, removed 92 μL supernatant from beads on magnet. Added 10 μL of 20 ng/μL trypsin working solution to each of samples 1-15 and incubated at 37° C. for 18.5 hours at 500 rpm. Removed 92 μL of each sample 10-12 from the beads. Acidified all the samples by adding 5 μL 10% TFA, checked pH (pH <3). Centrifuged at 15,000×g for 2 mins. Removed the following volumes for each sample groups: 65 μL for samples 1-3;

90 μL for samples 4-9; 82 μL for samples 10-15. Speed-vac dried the samples for about 1 hour. Added 0.2% FA and 4% ACN solution to each tube: 13 μL for samples 1-3 and 17 μL for all remaining samples. Stored all samples at −20° C. before nanoLC-MS/MS analysis.

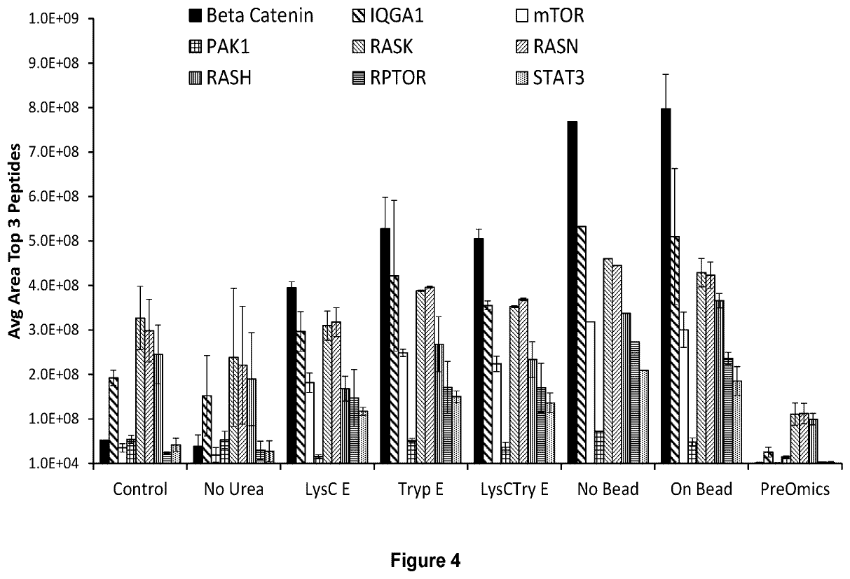

FIG. 4 shows the results of IP elution with enzyme and sequential reduction/alkylation. The results shown included the following: a) LysC elution b) Trypsin elution c) LysC/Trypsin elution d) Trypsin elution with On-bead reduction/alkylation (No Bead e) Trypsin elution with On-bead reduction/alkylation/digestion.

As shown in FIG. 4, the enzyme elution method showed better recovery of most of the targets compared to IP-MS elution buffer-based methods.

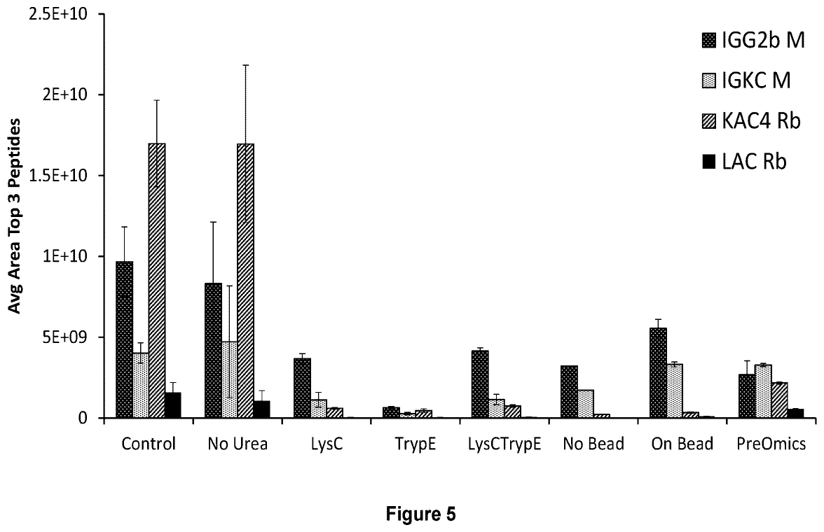

Significant reduction in antibody leaching was observed with enzyme elution method. As shown in FIG. 5, enzyme elution showed 1 to 2 orders of magnitude lower levels of IgGs compared to IP-MS elution buffer methods. Lower leaching of antibodies was found with the removal of bead after trypsin elution.

### Example 3—Single Pot Reduction/Alkylation Using Trypsin and/or LysC Enzyme Elution Method

An additional experiment was performed to evaluate various sample prep methods to decrease time/hands on/speed vac time by performing IP-MS using QC Mix Antibodies. The following were evaluated for IP elution with enzyme and one pot reduction/alkylation: a) LysC or Trypsin or LysC/Trypsin elution with one pot reduction/alkylation; b) LysC or Trypsin or

LysC/Trypsin elution with one pot reduction/alkylation with beads; c) LysC or Trypsin or LysC/Trypsin elution with one pot reduction/alkylation and digestion with beads.

Step 1. Prepared QC Mix for 15 IPs as shown in table 2 except replacement of Ras Antibody (PN #33-7200). Used 0.5 mg each of HCT116 (IGF Stim: Unstim)/HEK293 lysates for each IP reaction. Sealed the tubes with Parafilm and rotated overnight on 4° C.

Step 2. Used 50 μL beads for each IP using ratio of 1:5 for antibody amount to bead volume. Washed beads with 2× volume cold IP Lysis Buffer twice. Added 1 mL of antigen-antibody mixture to 50 μL beads and rotated for 1 hr at room temperature. Washed 3 times with 500 μL of Wash Buffer A followed by 2 times with 500 μL of Wash Buffer B. Evaluated the following methods for Mass Spec Sample Prep: Samples 1-3=Trypsin elution from bead; samples 4-6=Trypsin elution-Single Pot (SP) reduction/alkylation; samples 7-9=On bead digestion-Removed from bead then Trypsin (No Bead); samples 10-12=On bead digestion-Removed from bead then Trypsin-Single Pot reduction/alkylation (No bead-SP); samples 13-15=Control (Urea).

Step 3. Eluted samples 13-15 with 220 μL IP-MS Elution buffer, Vortexed well, quick spin and incubated for 10 minutes at room temperature, vortexed and quick spin and put on magnet then removed as much as possible into 2 mL tubes to pool the elutes together. Centrifuged the pooled samples at 15000× g for 2 minutes and put on magnet then pull elute divided into 205 μL per tube into different tubes labeled 13-15. Dried the samples in speed vac for about 1 hr (35° C.). Eluted samples 1-12 by adding 1 μg Trypsin in 100 μL 50 mM TEAB and incubated at 37° C. 800 rpm for 1 hour. After incubation, put samples 1-6 on magnet to remove beads and took 90 μl supernatant followed by addition of 2 μl 25 ng/μl GFP and 0.92 μl 0.5M TCEP. Incubated samples 1-3 at 60° C. for 30 minutes and samples 4-6 at 95° C. for 5 minutes. For samples 7-9, added 1 μl 0.5M TCEP and 2 μl 25 ng/μl GFP and incubated for 30 mins at 37° C. at 800 rpm. For samples 10-12, added 2 μL 25 ng/μL GFP and 25 μL one pot reduction/alkylation solution (Final 50 mM TEAB, pH 8.5; 10 mM TCEP; 20 mM chloracetyamide (CLAA)) and incubated at 95° C. 5 mins.

Step 4. Prepared the following solution for the next step of sample prep: Denaturation Solution—6M Urea+GFP-Used 360 mgs aliquots of Urea by adding 675 μL 50 mM TEAB and vortex (exothermic reaction). Added 400 μL of this solution to the GFP standard tubes 50 ng; 10 mM TCEP—Diluted 10 μL 0.5M TCEP with 490 μL 50 mM TEAB, pH8.5; 5 mM TCEP—Diluted 5 μL 0.5M TCEP with 495 μL 50 mM TEAB, pH8.5; 0.5 mM IAA—Dissolved No-weigh IAA 9.3 mgs in 100 μL MS-Grade water (Protect from light); 0.1 μg/μL Trypsin Stock—Dissolved 20 μg Trypsin protease (P #90057) in 200 μL 0.1% Acetic acid, stored as 30 μL aliquots at −80° C. 20 ng/μL trypsin working solution (prepared just before use)—Added 120 μL 50 mM TEAB solution to 30 μL aliquots of 0.1 μg/μL trypsin stock; 0.2% Formic Acid (FA), 4% Acetonitrile (ACN)—Added 940 μL MS grade water to 20 μL 10% FA and 40 μL of 100% ACN.

Step 5. For samples 13-15, added 10 μL 6M Urea/50 mM TEAB/rGFP solution to dried samples and vortexed for 30 seconds. Added 10 μL 10 mM TCEP mix and incubate at 35° C. mixing at 1000 rpm for 30 mins. Added 1 μL IAA for samples 13-15, 4.6 μL IAA for samples 1-3, 4.9 μL IAA for samples 7-9. Incubated all samples for 30 minutes at room temperature protected from light. Added 45 μL 50 mM TEAB, pH 8.5 to samples 13-15. For samples 7-12, removed supernatant from beads on magnet. Added 10 μL of 20 ng/μL trypsin working solution to each of samples 1-15 and incubated at 37° C. for 18.5 hours at 500 rpm. Acidified all the samples by adding 5 μL 10% TFA, checked pH (pH <3). Centrifuged at 15,000×g for 2 mins. Removed the following volumes for each sample groups: 96 μL for samples 1-3 and samples 7-9; 112 μL for samples 4-6 and samples 10-12. Speed-vac dried the samples for about 1 hour. Added 0.2% FA and 4% ACN solution to each tube: 17 μL for samples 1-12 and 13 μL for samples 13-15. Stored all samples at −20° C. before nanoLC-MS/MS analysis.

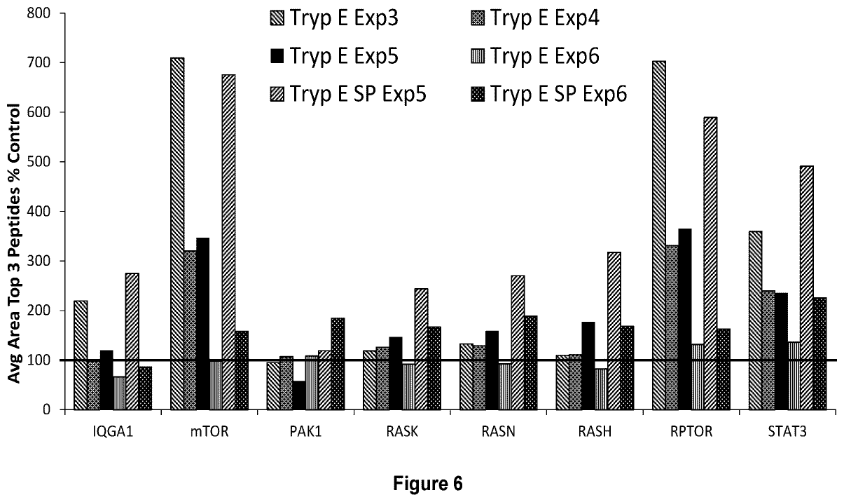

As shown in FIG. 6, enzyme elution with the single pot reduction/alkylation showed better recovery of targets from QC mixture.

### Example 4

An additional experiment was conducted to evaluate various sample prep methods to further validate enzyme elutions by performing IP-MS using QC Mix Antibodies.

Step 1. Prepared QC Mix for 12 IPs as shown in table 2 except replacement of Ras Antibody (PN #33-7200). Used 0.5 mg each of HCT116 (IGF Stim: Unstim)/HEK293 lysates for each IP reaction. Sealed the tubes with Parafilm and rotated overnight on 4° C.

Step 2. Used 50 μL beads for each IP using ratio of 1:5 for antibody amount to bead volume. Washed beads with 2× volume cold IP Lysis Buffer twice. Added 1 mL of antigen-antibody mixture to 50 μL beads and rotated for 1 hr at room temperature. Washed 3 times with 500 μL of Wash Buffer A followed by 2 times with 500 μL of Wash Buffer B. Evaluated the following methods for Mass Spec Sample Prep: Samples 1-3=Control (Urea); Samples 4-6=Trypsin elution from bead; samples 7-9=Trypsin elution from beads-Single Pot (SP) reduction/alkylation; samples 10-12=On bead digestion-Removed from bead then Trypsin.

Step 3. Eluted samples 1-3 with 220 μL IP-MS Elution buffer, Vortexed well, quick spin and incubated for 10 minutes at room temperature, vortexed and quick spin and put on magnet then removed as much as possible into 1.5 mL tubes to pool the elutes together. Centrifuged the pooled samples at 15000×g for 2 minutes and put on magnet then aliquoted into 205 μL per tube into different tubes labeled 1-3. Dried the samples in speed vac for about 1 hr (35° C.). Eluted samples 4-12 by adding 1 μg Trypsin in 100 μL 50 mM TEAB and incubated at 37° C. 800 rpm for 1 hour. After incubation, put samples 4-6 on magnet to remove beads and took 90 μl supernatant followed by addition of 2 μl 25 ng/μl GFP and 0.92 μl 0.5M TCEP. Incubated samples 4-6 at 60° C. for 30 minutes. After incubation, put samples 7-9 on magnet to remove beads and took 90 μl supernatant followed by addition of 2 μl 25 ng/μl GFP and 25 μL one pot reduction/alkylation solution (25 μL one pot reduction/alkylation solution (Final 50 mM TEAB, pH 8.5; 10 mM TCEP; 20 mM chloracetyamide (CLAA)) and incubated at 95° C. for 5 minutes. For samples 10-12, added 1 μl 0.5M TCEP and 2 μl 25 ng/μl GFP and incubated for 30 mins at 37° C. at 800 rpm.

Step 4. Prepared the following solution for the next step of sample prep: Denaturation Solution—6M Urea+GFP-Used 360 mgs aliquots of Urea by adding 675 μL 50 mM TEAB and vortex (exothermic reaction). Added 400 μL of this solution to the GFP standard tubes 50 ng; 10 mM TCEP—Diluted 10 μL 0.5M TCEP with 490 μL 50 mM TEAB, pH8.5; 5 mM TCEP—Diluted 5 μL 0.5M TCEP with 495 μL 50 mM TEAB, pH8.5; 0.5 mM IAA—Dissolved No-weigh IAA 9.3 mgs in 100 μL MS-Grade water (Protect from light); 0.1 μg/μL Trypsin Stock—Dissolved 20 μg Trypsin protease (P #90057) in 200 μL 0.1% Acetic acid, stored as 30 μL aliquots at −80° C. 20 ng/μL trypsin working solution (prepared just before use)—Added 120 μL 50 mM TEAB solution to 30 μL aliquots of 0.1 μg/μL trypsin stock; 0.2% Formic Acid (FA), 4% Acetonitrile (ACN)—Added 940 μL MS grade water to 20 μL 10% FA and 40 μL of 100% ACN.

Step 5. For samples 1-3, added 10 μL 6M Urea/50 mM TEAB/rGFP solution to dried samples and vortexed for 30 seconds. Added 10 μL 10 mM TCEP mix and incubate at 35° C. mixing at 1000 rpm for 30 mins. Added 1 μL IAA for samples 1-3, 4.6 μL IAA for samples 4-6, 4.9 μL IAA for samples 10-12. Incubated all samples for 30 minutes at room temperature protected from light. Added 45 μL 50 mM TEAB, pH 8.5 to samples 13-15. For samples 7-12, removed supernatant from beads on magnet. Added 10 μL of 20 ng/μL trypsin working solution to each of samples 1-12 and incubated at 37° C. for 18.5 hours at 500 rpm. Acidified all the samples by adding 5 μL 10% TFA, checked pH (pH <3). Centrifuged at 15,000×g for 2 mins. Removed the following volumes for each sample groups: 68 μL for samples 1-3; 96 μL for samples 4-6 and samples 10-12; 112 μL for samples 7-9. Speed-vac dried the samples for about 1 hour. Added 0.2% FA and 4% ACN solution to each tube: 13 μL for samples 1-3 and 17 μL for samples 4-12. Stored all samples at −20° C. before nanoLC-MS/MS analysis.

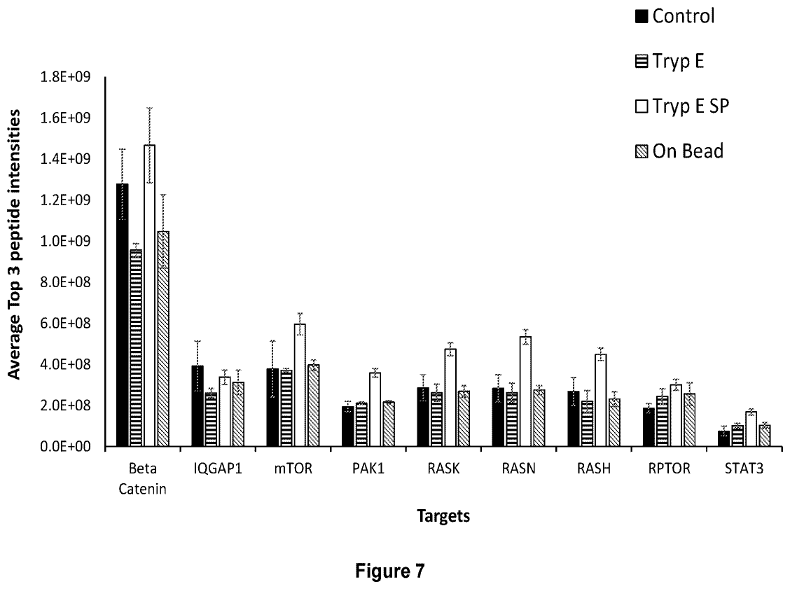

The results are shown in FIG. 7. Trypsin elution with single pot (SP) reduction/alkylation showed equal or better recovery of 9 target proteins compared to Urea, Trypsin elution with sequential reduction/alkylation and on bead trypsin digestion.

### Example 5

An experiment was performed to evaluate sample prep methods by performing IP-MS using Akt Phospho Mix Antibodies (Thermo Fisher Scientific, PN #A40086).

Step 1. Prepared AKT Phospho Antibody mixture for 9 IPs. Used 1 mg each of MCF7 Stim (+hIGF) lysate for each IP reaction. Sealed the tubes with Parafilm and rotated overnight on 4° C.

Step 2. Used 55 μL beads for each IP using ratio of 1:5 for antibody amount to bead volume. Washed beads with 2× volume cold IP Lysis Buffer twice. Added 1 mL of antigen-antibody mixture to 55 μL beads and rotated for 1 hr at room temperature. Washed 3 times with 500 μL of Wash Buffer A followed by 2 times with 500 μL of Wash Buffer B. Evaluated the following methods for Mass Spec Sample Prep: Samples 1-3=Control (Urea); Samples 4-6=Trypsin elution from bead; samples 7-9=Trypsin elution from beads-Single Pot (SP) reduction/alkylation.

Step 3. Eluted samples 1-3 with 220 μL IP-MS Elution buffer, Vortexed well, quick spin and incubated for 10 minutes at room temperature, vortexed and quick spin and put on magnet then removed as much as possible into 1.5 mL tubes to pool the elutes together. Centrifuged the pooled samples at 15000×g for 2 minutes and put on magnet then aliquoted into 205 μL per tube into different tubes labeled 1-3. Dried the samples in speed vac for about 1 hr (35° C.). Eluted samples 4-9 by adding 1 μg Trypsin in 100 μL 50 mM TEAB and incubated at 37° C. 800 rpm for 1 hour. After incubation, put samples 4-6 on magnet to remove beads and took 90 μl supernatant followed by addition of 2 μl 25 ng/μl GFP and 0.92 μl 0.5M TCEP. Incubated samples 4-6 at 60° C. for 30 minutes. After incubation, put samples 7-9 on magnet to remove beads and took 90 μl supernatant followed by addition of 2 μl 25 ng/μl GFP and 25 μL one pot reduction/alkylation solution (25 μL one pot reduction/alkylation solution (Final 50 mM TEAB, pH 8.5; 10 mM TCEP; 20 mM chloracetyamide (CLAA)) and incubated at 95° C. for 5 minutes.

Step 4. Prepared the following solution for the next step of sample prep: Denaturation Solution—6M Urea+GFP-Used 360 mgs aliquots of Urea by adding 675 μL 50 mM TEAB and vortex (exothermic reaction). Added 400 μL of this solution to the GFP standard tubes 50 ng; 10 mM TCEP—Diluted 10 μL 0.5M TCEP with 490 μL 50 mM TEAB, pH8.5; 5 mM TCEP—Diluted 5 μL 0.5M TCEP with 495 μL 50 mM TEAB, pH8.5; 0.5 mM IAA—Dissolved No-weigh IAA 9.3 mgs in 100 μL MS-Grade water (Protect from light); 0.2 μg/μL Trypsin Stock—Dissolved 20 μg Trypsin protease (P #90057) in 100 μL 0.1% Acetic acid, stored as 30 μL aliquots at −80° C. 40 ng/μL trypsin working solution (prepared just before use)—Added 120 μL 50 mM TEAB solution to 30 μL aliquots of 0.2 μg/μL trypsin stock; 0.2% Formic Acid (FA), 4% Acetonitrile (ACN)—Added 940 μL MS grade water to 20 μL 10% FA and 40 μL of 100% ACN.

Step 5. For samples 1-3, added 10 μL 6M Urea/50 mM TEAB/rGFP solution to dried samples and vortexed for 30 seconds. Added 10 μL 10 mM TCEP mix and incubate at 35° C. mixing at 1000 rpm for 30 mins. Added 1 μL IAA for samples 1-3 and 4.6 μL IAA for samples 4-6. Incubated all samples for 30 minutes at room temperature protected from light. Added 45 μL 50 mM TEAB, pH 8.5 to samples 13-15. For samples 7-9, removed supernatant from beads on magnet. Added 10 μL of 20 ng/μL trypsin working solution to each of samples 1-9 and incubated at 37° C. for 18.5 hours at 500 rpm. Acidified all the samples by adding 5 μL 10% TFA, checked pH (pH <3). Centrifuged at 15,000×g for 2 mins. Removed the following volumes for each sample groups: 68 μL for samples 1-3; 96 μL for samples 4-6; 112 μL for samples 7-9. Speed-vac dried the samples for about 1 hour. Added 0.2% FA and 4% ACN solution to each tube: 13 μL for samples 1-3 and 17 μL for samples 4-9. Stored all samples at −20° C. before nanoLC-MS/MS analysis.

The results are shown in FIG. 8. Trypsin elution with single pot (SP) reduction/alkylation showed better recovery of 9 of 11 AKT pathway phosphorylated proteins compared to control Urea and Trypsin elution with sequential reduction/alkylation methods.

Table 3 shows day to day coefficient of variation (CV) s are better for enzyme elution.

TABLE 3
% CV between different experiments (days)

| Target | Urea | TrypE | TrypE_SP |
|---|---|---|---|
| pAKT2 | 35% | 4% | 13% |
| pAKT1 | 57% | 22% | 9% |
| pPRAS40 | 15% | 6% | 8% |
| pmTOR | 38% | 10% | 7% |
| pGSK3a | 22% | 7% | 4% |
| pIGF1R | 34% | 2% | 16% |
| pIRS1 | 20% | 12% | 40% |
| pp70S6K | 15% | 16% | 16% |
| pGSK3b | 24% | 15% | 5% |
| pTSC2 | 15% | 20% | 5% |
| pPTEN | 25% | 141% | 61% |

In view of the above, the optimal condition was found to be trypsin elution from the beads, combined with single pot reduction/alkylation followed by overnight second trypsin digestion.

### Example 6—Optimizing Time for Second Trypsin Digestion

Experiments were set up to test different digestion times for the second trypsin digestion for MS sample prep optimization. The emphasis was to compare the overnight second trypsin digestion with shorter digestion times with purpose to produce a one-day sample prep method.

### Experiment Protocol:

Step 1. Materials used are same as listed in Table 1. AKT Phospho Multiplex Antibody mix (PN #A40086) was used and mixed with 0.5 mg each HCT116 (+/−) lysates. Performed total of 16 IP reactions (2 replicates for each condition) by adding appropriate amount of antibody mix in 1.5 mL low-protein binding tube. Parafilmed each tube and incubated overnight at 4° C. mixing on a rotator. After incubation added streptavidin magnetic beads using ratio of 1:5 (Antibody: Beads, Since 14 μg Antibody was used 70 μL beads were added per 1 mL). Washed beads with 2× volume cold IP Lysis Buffer twice. Removed wash buffer then added back original volume of IP Lysis Buffer to each tube then added washed beads to each IP. Mixed antibody/antigen samples and rotated for 1 hour at room temperature. Washed 3 times with 500 μL Wash Buffer A. Washed 2 times with 500 μL Wash Buffer B.

Step 2. The following MS sample prep solutions were prepared: 50 mM TEAB—Diluted 1M Trimethylammonium Bicarbonate (TEAB) (PN #90114) to 50 mM by adding 0.5 mL TEAB in 9.5 mL MS grade water, pH 8.5; Single Pot Reduction Alkylation—Final 50 mM TEAB pH 8.5; 10 mM TCEP; 20 mM chloroacetamide (CLAA); 0.2 μg/μL Trypsin Stock—Dissolved 20 μg Trypsin protease (PN #90057) in 100 μL 0.1% Acetic acid; CaCl₂ solution—Add 13.78 mg CaCl₂ in 150 μL Trypsin+50 mM TEAB Solution; 0.2% Formic Acid (FA), 4% Acetonitrile (ACN)—Add 940 μL MS grade water, 20 μL 10% FA and 40 μL of 100% ACN.

Step 3. Mass Spec Sample Prep Various Methods tested included. Samples 1-2=Control-Regular overnight trypsin digestion at 37° C.; samples 3-4=1 hour trypsin digestion at 37° C.; samples 5-6=2 hour trypsin digestion at 37° C.; samples 7-8=3 hour trypsin digestion at 37° C.; samples 9-10=4 hour trypsin digestion at 37° C.; samples 11-12=1 hour trypsin digestion at 60° C.; samples 13-14=2 hour trypsin digestion at 60° C.; and samples 15-16=2 hour trypsin digestion at 60° C.+50 mM CaCl₂. All reactions were performed in Thermomixer set at stated temperature and 800 rpm.

Step 4. The sample prep continued for trypsin elution by adding 1 μg trypsin in 100 μl 50 mM TEAB to each sample and incubated for 1 hour at 37° C. in a thermomixer shaking at 800 rpm. After 1 HR incubation, tubes were placed onto magnet to remove beads. 90 μL of supernatant was removed and 2 μL 25 ng/μL GFP and 25 μl single pot reduction/alkylation solution were added to each reaction and incubated at 95° C. for 5 mins.

Step 5. After Reduction/Alkylation added trypsin digestion enzymes as stated in step 3 and incubated at stated times and temperatures for digestion reactions.

Step 6. Acidified the samples by adding 3.5 μL 10% TFA (pH <3) to all samples except 15-16 where 4.5 μL of 10% TFA+1 μL 25% TFA was added acidify samples. Centrifuged all samples at 15,000×g for 2 mins and then removed 112 μL of samples to a clean 1.5 ml low protein binding tube. Dried down the samples for about 1 hr in speed vac. Added 17 μL of 0.2% FA and 4% ACN to each tube. Stored all samples at −20° C. before nanoLC-MS/MS analysis.

Results of three experiments are summarized in Table 4 below.

TABLE 4

| Exp 1 n = 3 | Exp 2 n = 2 | Exp 3 n = 3 |
|---|---|---|
| (Operator A) | (Operator A) | (Operator B) |
| 1 Hour at 37° C. | 1 Hour at 37° C. | 1 Hour at 37° C. |
| | 1 Hour at 60° C. | |
| 2 Hours at 37° C. | 2 Hours at 37° C. | 2 Hours at 37° C. |
| | 2 Hours at 60° C. | |
| | 2 Hours at 60° C. + | |
| | 50 mM CaCl2 | |
| 3 Hours at 37° C. | 3 Hours at 37° C. | 3 Hours at 37° C. |
| 4 Hours at 37° C. | 4 Hours at 37° C. | 4 Hours at 37° C. |
| Overnight (18.5 Hrs) | Overnight (18.5 Hrs) | Overnight (18.5 Hrs) |
| at 37° C. (Control) | at 37° C. (Control) | at 37° C. (Control) |

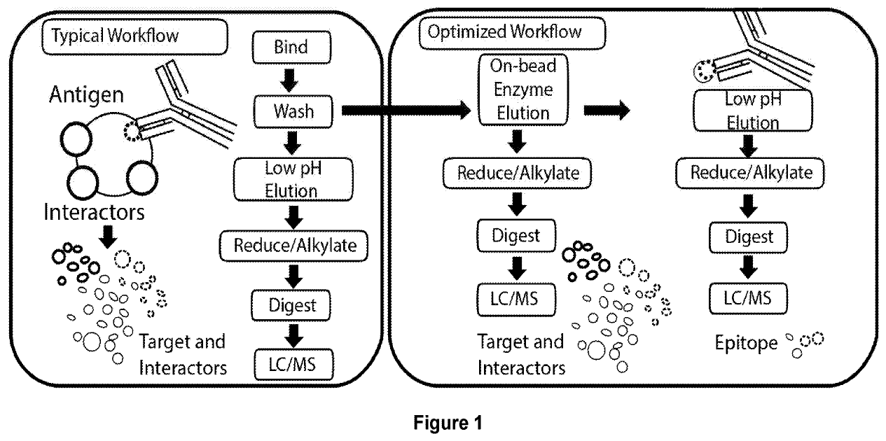

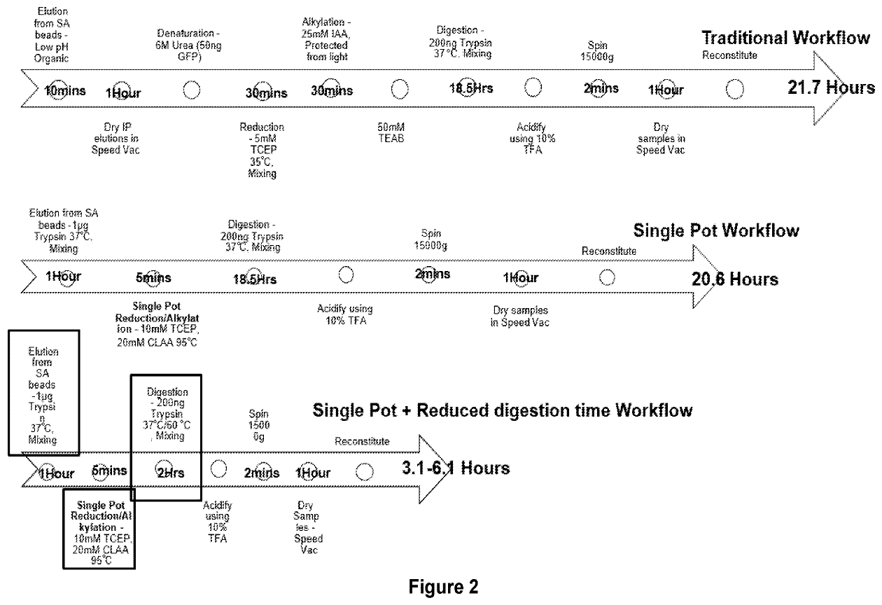

As shown in FIGS. 9-11, 1, 2, and 3 hours showed very comparable data compared to overnight digestion.

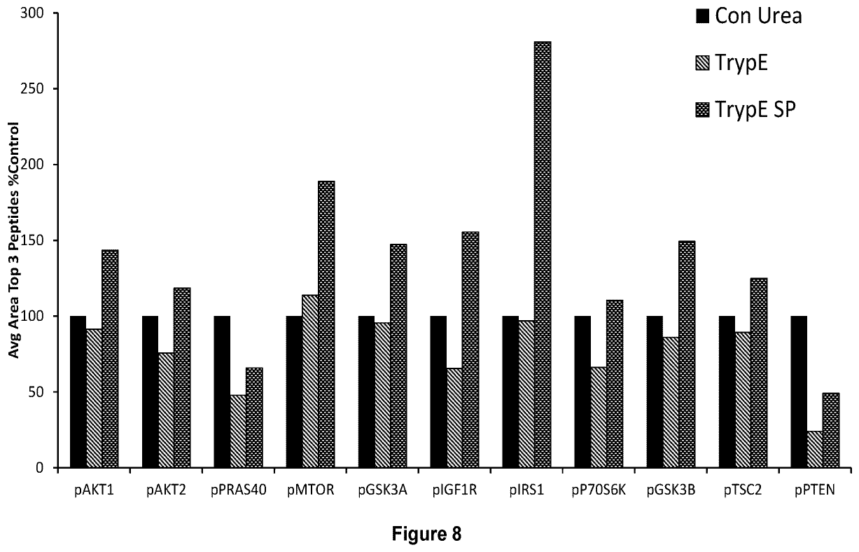

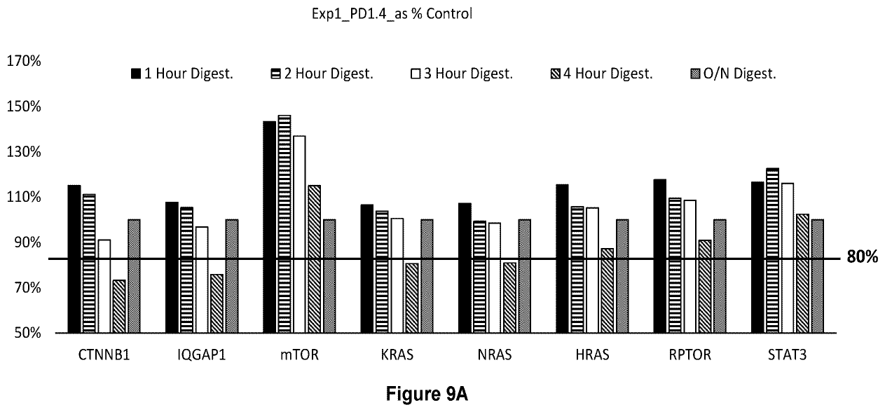

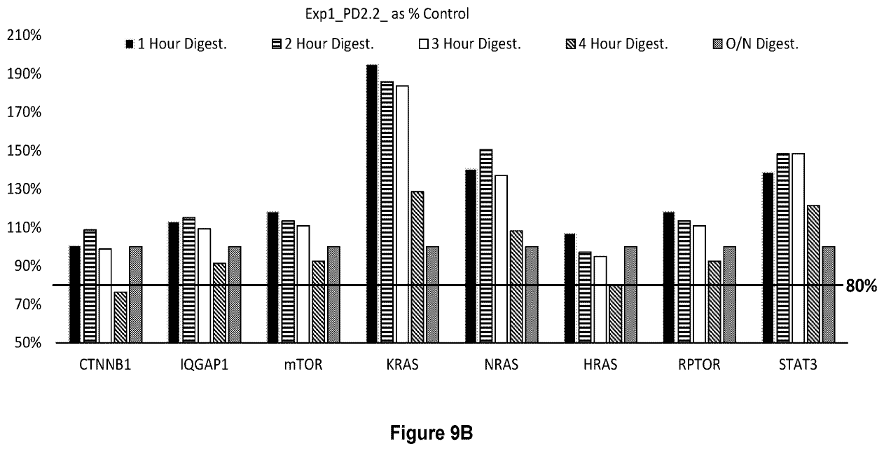

FIGS. 9A and 9B show average top peptide areas from Experiment 1 above. Samples digested for 1/⅔ hours give equivalent or better intensities for all the targets as compared to those digested overnight. Samples digested for 4 hours showed lower intensities. PD 1.4 and 2.2 results correlate.

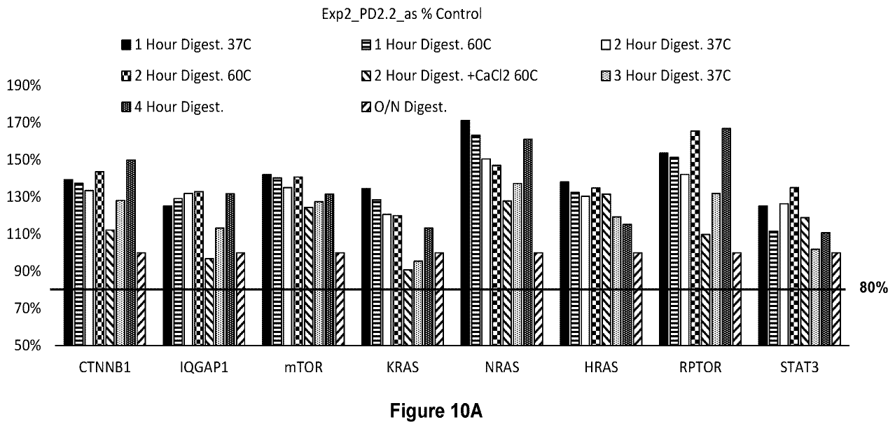

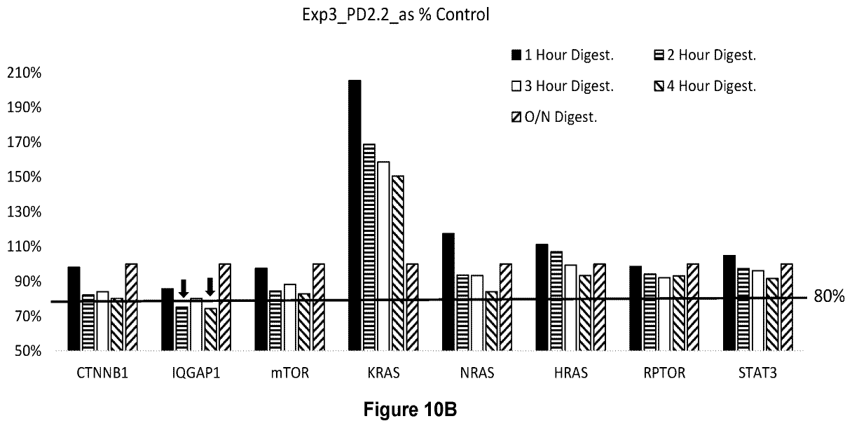

FIG. 10A shows average peptide areas for Experiment 2 above. Samples digested for 1/⅔ hours give equivalent or better intensities for all the targets as compared to those digested overnight. Samples digested for 4 hours showed comparable intensities.

FIG. 10B shows average peptide areas for Experiment 3 above. Except for IQGAP1, all the targets meet the specs for all conditions.

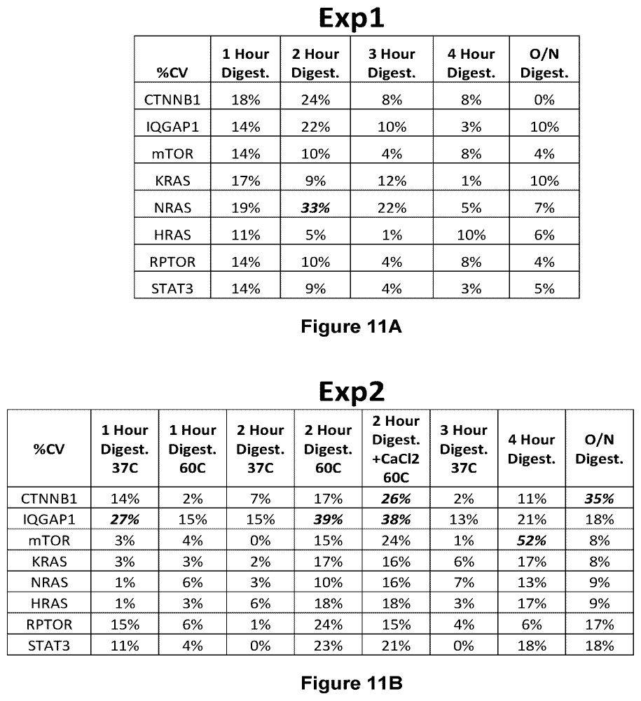

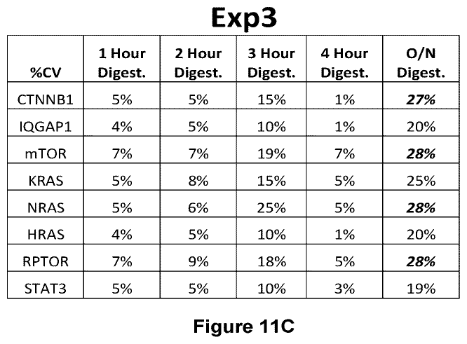

FIG. 11 shows % CV of peptide area for three experiments shown in Table 4 above. Overall <25% CV was observed with 1/2/3/4 hrs or O/N digestion time points.

Increasing the temperature to 60° C. and addition of CaCl₂) did not improve results.

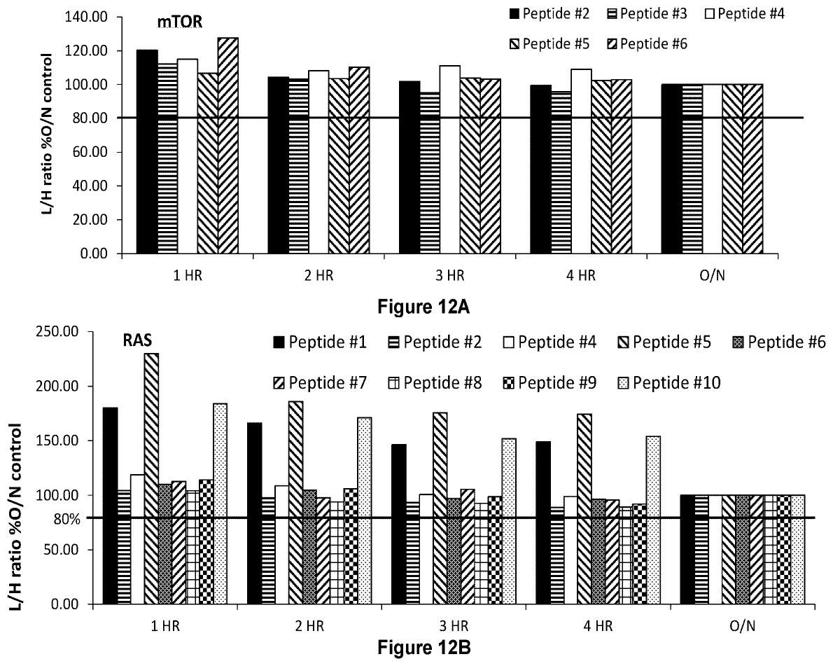

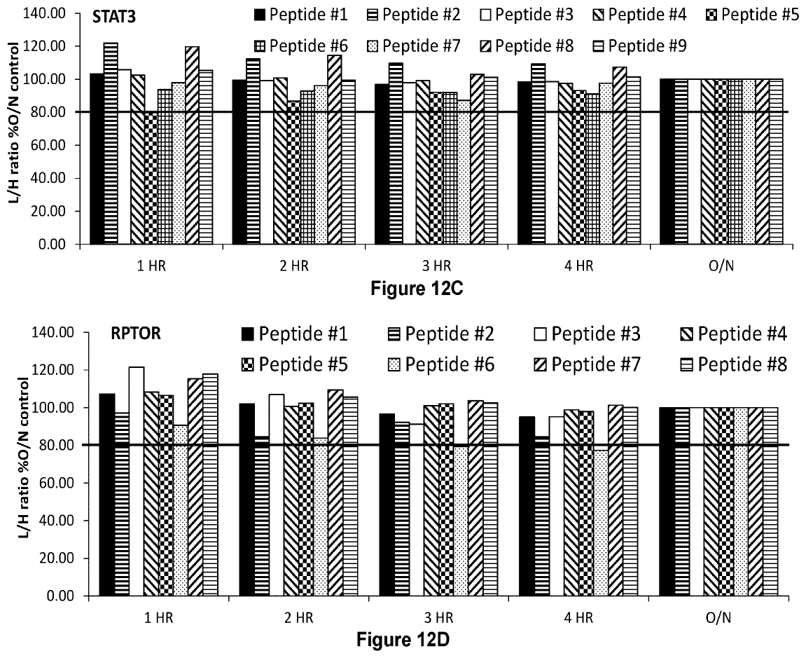

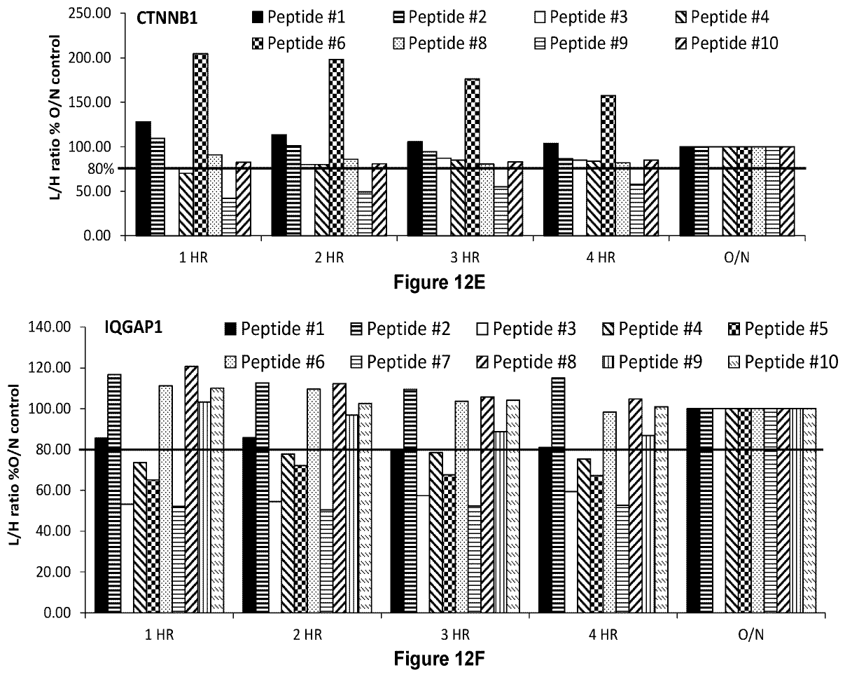

Targeted MS analysis was performed to evaluate recovery of multiple unique peptides for each target protein across different digestion times. The results are shown in FIGS. 12A-F. Low recovery of most peptides for CTNNB1 (FIG. 12E) and IQGAP1 (FIG. 12 F) was observed with different digestion time points.

### Example 7—Effects of Trypsin Amount and Testing with LysC in Second Digestion

Experiments were performed to test different trypsin amounts with different digestion times with and without LysC for MS sample prep optimization. IP was followed by MS (Single pot reduction/alkylation) and modified trypsin digestion step. Table 5 below shows the experimental design.

TABLE 5

| One Hour at 37° C. | Two Hour at 37° C. | Overnight at 37° C. |
|---|---|---|
| 200 ng Trypsin | 200 ng Trypsin | 200 ng Trypsin |
| (1:40) | (1:40) | (1:40) |
| 600 ng Trypsin | 600 ng Trypsin | — |
| (1:13) | (1:13) | |
| 300 ng Trypsin + | 300 ng Trypsin + | |
| 300 ng LysC (1:13) | 300 ng LysC (1:13) | |
| 800 ng Trypsin | 800 ng Trypsin | |
| (1:10) | (1:10) | |

### Experiment Protocol:

Step 1. Materials used are same as listed in Table 1. Prepared biotinylated antibody mixture as shown in Table 2 above and mixed with 0.5 mg each of HCT116 (IGF Stim: Unstim)/HEK293 lysates. Performed total of 24 IP reactions by adding appropriate amount of antibody mix in 1.5 mL low protein binding tube. Parafilmed each tube and incubated overnight at 4° C. mixing on a rotator. After incubation added streptavidin magnetic beads using ratio of 1:5 (Antibody: Beads, Since 10 μg Antibody was used 50 μL beads were added per 1 mL). Washed beads with 2× volume cold IP Lysis Buffer twice. Removed wash buffer then added back original volume of IP Lysis Buffer to each tube. Pooled all IPs together in a 50 ml conical tube and aliquoted into 25 1 mL tubes and then added washed beads to each IP. Mixed antibody/antigen samples and rotated for 1 hour at room temperature. Washed 3 times with 500 μL Wash Buffer A. Washed 2 times with 500 μL Wash Buffer B.

Step 2. The following MS sample prep solutions were prepared: 50 mM TEAB—Diluted 1M Trimethylammonium Bicarbonate (TEAB) (PN #90114) to 50 mM by adding 0.5 mL TEAB in 9.5 mL MS grade water, pH 8.5; Single Pot Reduction Alkylation—Final 50 mM TEAB pH 8.5; 10 mM TCEP; 20 mM chloroacetamide (CLAA); 0.2 μg/μL Trypsin Stock—Dissolved 20 μg Trypsin protease (PN #90057) in 100 μL 0.1% Acetic acid; Various trypsin stocks—20 ng/μL=30 μL trypsin stock+120 μL 50 mM TEAB solution then add 10 μL to sample 1, 2, 9, 10, 17, 18, 19-24; 60 ng/μL=90 μL trypsin stock+60 μL 50 mM TEAB solution then add 10 μL to samples 3, 4, 11 and 12; 80 ng/μL=120 μL trypsin stock+30 μL 50 mM TEAB solution then add 10 μL to samples 7, 8, 15 and 16; 60 ng/μL Trypsin/LysC stock-Add 200 μL 50 mM Acetic acid to 20 μg trypsin/LysC then make solution=90 μL trypsin/LysC stock+60 μL 50 mM TEAB solution then add 10 μL to samples 5, 6, 13 and 14; 0.2% Formic Acid (FA), 4% Acetonitrile (ACN)—Add 940 μL MS grade water, 20 μL 10% FA and 40 μL of 100% ACN.

Step 3. Mass Spec Sample Prep Various Methods tested included. Samples 1-2=1 hr trypsin digestion at 37° C., 200 ng trypsin (˜1:40, assuming we have 6-8 μg peptides); samples 3-4=1 hr trypsin digestion at 37° C., 600 ng trypsin (˜1:10); samples 5-6=1 hr trypsin digestion at 37° C., 300 ng trypsin+300 ng LysC (˜1:10); samples 7-8=1 hr trypsin digestion at 37° C., 800 ng trypsin (˜1:10); samples 9-10=2 hr trypsin digestion at 37° C., 200 ng trypsin; samples 11-12=2 hr trypsin digestion at 37° C., 600 ng trypsin; samples 13-14=2 hr trypsin digestion at 37° C., 300 ng trypsin+300 ng LysC; samples 15-16=2 hr trypsin digestion at 37° C., 800 ng trypsin; samples 17-18=Overnight trypsin digestion at 37° C., 200 ng trypsin; samples 19-21=2 hr trypsin digestion at 37° C., 200 ng trypsin (extra for Peptide Assay); and samples 22-24=overnight trypsin digestion at 37° C., 200 ng trypsin.

Step 4. The sample prep continued for trypsin elution by adding 1 μg trypsin in 100 μl 50 mM TEAB to each sample and incubated for 1 hour at 37° C. in a thermomixer shaking at 800 rpm.

Step 5. After 1 HR incubation tubes were placed onto magnet to remove beads and 90 μL of supernatant was removed and 2 μL 25 ng/μL GFP and 25 μl single pot reduction/alkylation solution were added to each reaction and incubated at 95° C. for 5 mins.

Step 6. After Reduction/Alkylation added digestion enzymes as stated in step 3 and incubated at stated times and temperatures for digestion reactions.

Step 7. Acidified the samples by adding 4.5 μL 10% TFA+1 μL 25% TFA to acidify samples (pH <3). Centrifuged all samples at 15,000×g for 2 mins and then removed 112 μL of samples and transferred to clean 1.5 ml low protein binding tube. Dried down the samples for about 1 hr in speed vac. Added 17 μL of QC peptide Mix (80 fmol) in 0.2% FA and 4% ACN to samples 1-18. Reconstitute samples 19-24 in 17 μL 4% ACN and MS grade water. Stored all samples at −20° C. before nanoLC-MS/MS analysis.

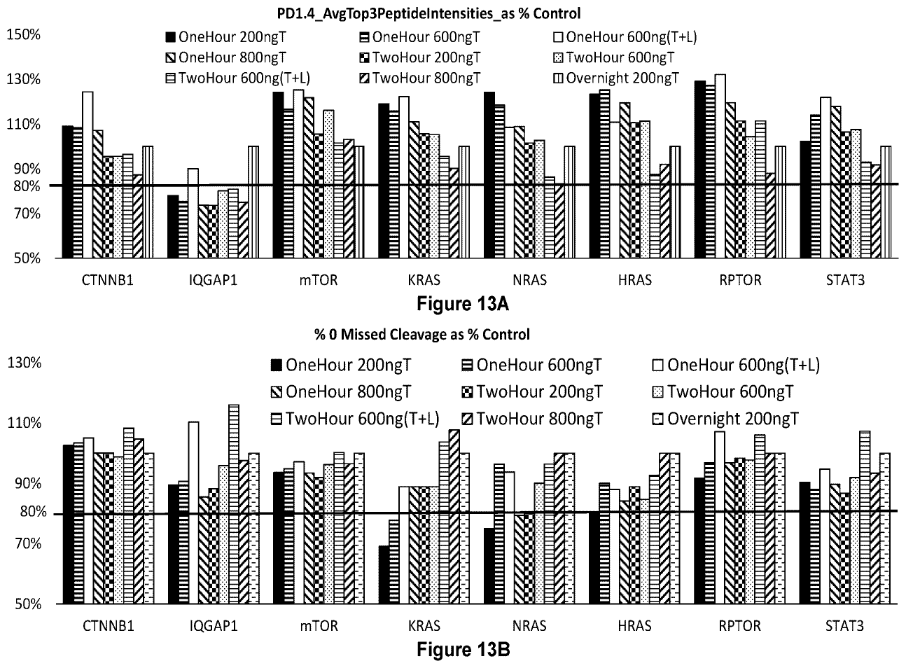

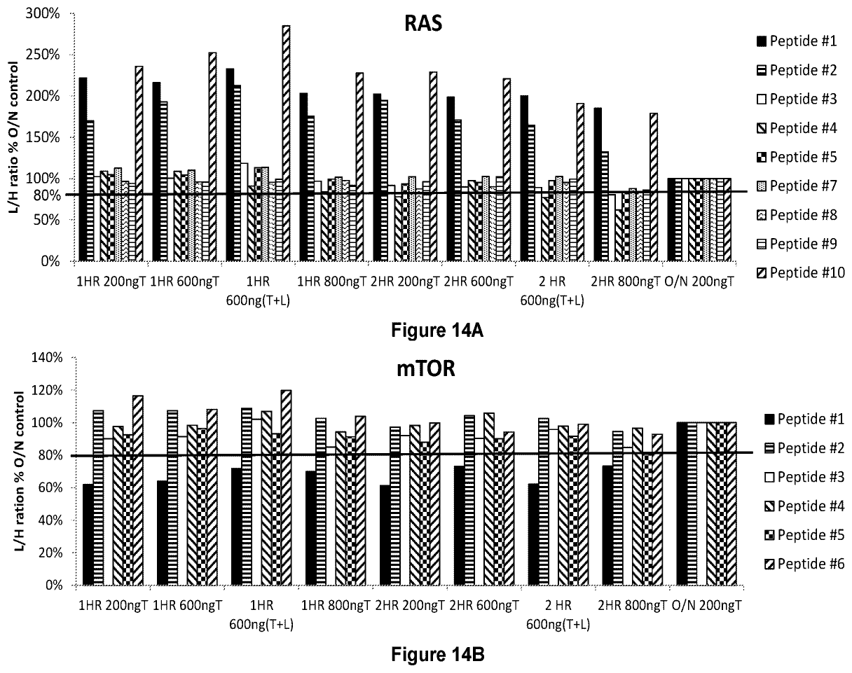

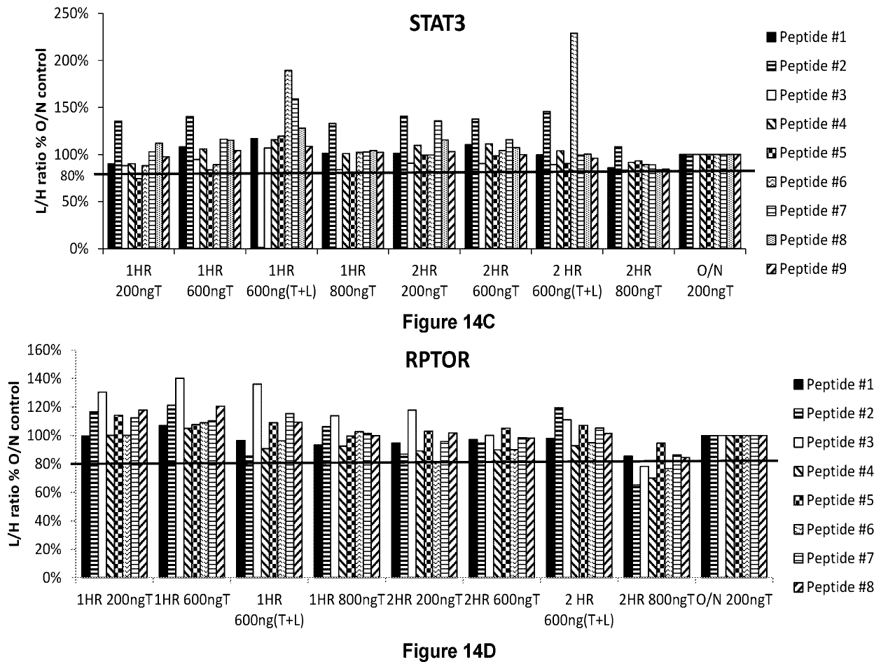

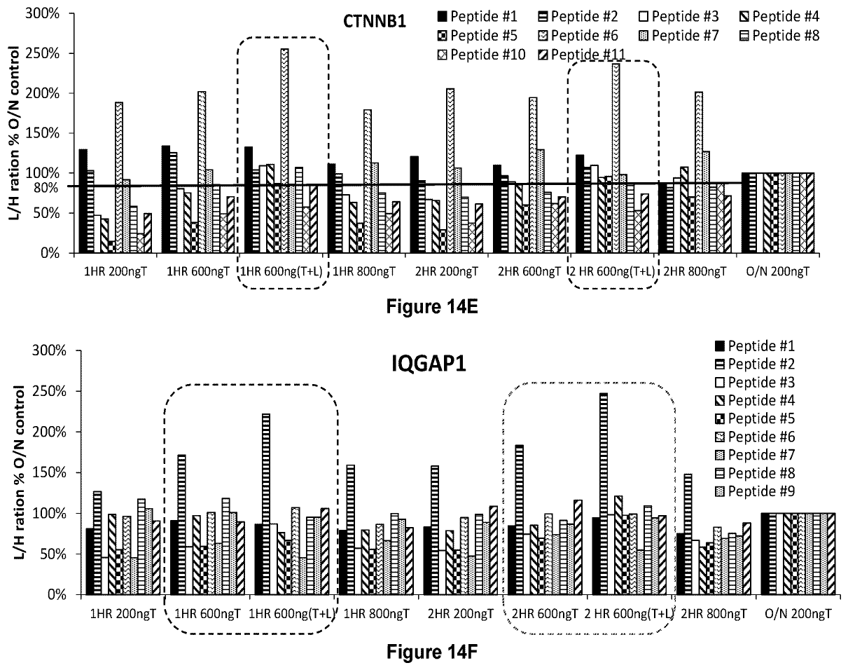

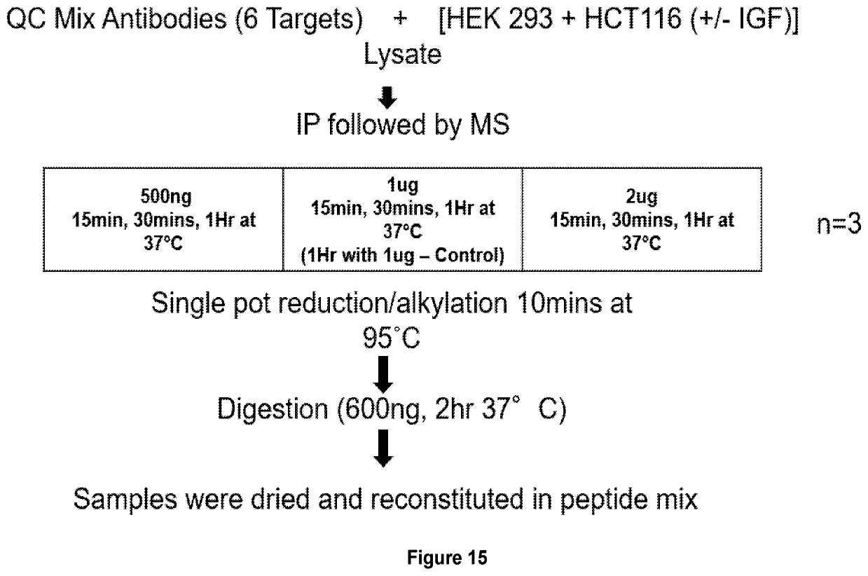

Results are shown in FIGS. 13-14. In FIG. 13A, for peptide area: 1 hr with Trypsin/LysC combination passed specs (<20%) for all targets. In FIG. 13B, 0% missed cleavage peptides: All targets passed specs across all conditions except KRAS/NRAS at 1 hr.

The results of targeted analysis are shown in FIGS. 14A-F. FIGS. 14A-B show no significant difference found with different amount of trypsin or trypsin/LysC combination. FIGS. 14C and D show no significant difference found with different amount of trypsin or trypsin/LysC combo except 2 hr with 800 ng trypsin. FIGS. 14E-F show better recovery of most peptides for CTNNB1 and IQGAP1 observed with more trypsin (600-800 ngs) or trypsin/LysC combo (600 ng).

### Example 8—Optimization of Enzymatic IP Elution

Experiments were performed to test trypsin elution from beads by varying enzyme amounts and elution times. A flowchart is shown in FIG. 1.

### Experimental Protocol:

Step 1. Materials used are same as listed in Table 1. Prepared biotinylated antibody mixture as shown in Table 2 above and mixed with 0.5 mg each of HCT116 (IGF Stim: Unstim)/HEK293 lysates. Performed total of 27 IP reactions by adding appropriate amount of antibody mix in 1.5 mL low protein binding tube. Parafilmed each tube and incubated overnight at 4° C. mixing on a rotator. After incubation added streptavidin magnetic beads using ratio of 1:5 (Antibody: Beads, Since 10 μg Antibody was used 50 μL beads were added per 1 mL). Washed beads with 2× volume cold IP Lysis Buffer twice. Removed wash buffer then added back original volume of IP Lysis Buffer to each tube. Pooled all IPs together in a 50 ml conical tube and aliquoted into 25 1 mL tubes and then added washed beads to each IP. Mixed antibody/antigen samples and rotated for 1 hour at room temperature. Washed 3 times with 500 μL Wash Buffer A. Washed 2 times with 500 μL Wash Buffer B.

Step 2. The following MS sample prep solutions were prepared: 50 mM TEAB—Diluted 1M Trimethylammonium Bicarbonate (TEAB) (PN #90114) to 50 mM by adding 0.5 mL TEAB in 9.5 mL MS grade water, pH 8.5; Single Pot Reduction Alkylation—Final 50 mM TEAB pH 8.5; 10 mM TCEP; 20 mM chloroacetamide (CLAA); 0.2 μg/μL Trypsin Stock—Dissolved 20 μg Trypsin protease (PN #90057) in 100 μL 0.1% Acetic acid; Various trypsin stocks—1 μg trypsin=5 μL 0.2 μg/μL trypsin stock+95 μL 50 mM TEAB solution then add 10 μL to sample 1-9; 2 μg trypsin=10 μL 0.2 μg/μL trypsin stock+90 μL 50 mM TEAB solution then add 10 μL to sample 10-18; 0.5 μg trypsin=2.5 μL 0.2 μg/μL trypsin stock+97.5 μL 50 mM TEAB solution then add 10 μL to sample 19-27; 0.2% Formic Acid (FA), 4% Acetonitrile (ACN)—Add 940 μL MS grade water, 20 μL 10% FA and 40 μL of 100% ACN.

Step 3. Mass Spec Sample Prep Various Methods tested included. Samples 1-3=1 μg trypsin digestion 1 hour at 37° C.; samples 4-6=1 μg trypsin digestion 30 min at 37° C.; samples 7-9=1 μg trypsin digestion 15 min at 37° C.; samples 10-12=2 μg trypsin digestion 1 hour at 37° C.; samples 13-15=2 μg trypsin digestion 30 min at 37° C.; samples 16-18=2 μg trypsin digestion 15 min at 37° C.; samples 19-21=0.5 μg trypsin digestion 1 hour at 37° C.; samples 22-24=0.5 μg trypsin digestion 30 min at 37° C.; samples 25-27=2 μg trypsin digestion 15 min at 37° C.

Step 4. The sample prep continued for trypsin elution by adding 0.5, 1, or 2 μg trypsin in 100 μl 50 mM TEAB to each sample and incubated for 15 min, 30 min, or 1 hour at 37° C. in a thermomixer shaking at 800 rpm.

Step 5. After 1 HR incubation tubes were placed onto magnet to remove beads and 90 μL of supernatant was removed and 2 μL 25 ng/μL GFP and 25 μl single pot reduction/alkylation solution were added to each reaction and incubated at 95° C. for 5 mins.

Step 6. After Reduction/Alkylation 1 μg trypsin was added in 50 mM TEAB and incubated for 2 hours at 37° C. shaking at 800 rpm.

Step 7. Acidified the samples by adding 2.5 μL 25% TFA to acidify samples (pH <3). Centrifuged all samples at 15,000×g for 2 mins and then removed 112 μL of samples and transferred to clean 1.5 ml low protein binding tube. Dried down the samples for about 1 hr in speed vac. Added 20 μL of QC peptide Mix (80 fmol) in 0.2% FA and 4% ACN to samples 1-18. Reconstitute samples 19-24 in 17 μL 4% ACN and MS grade water. Stored all samples at −20° C. before nanoLC-MS/MS analysis.

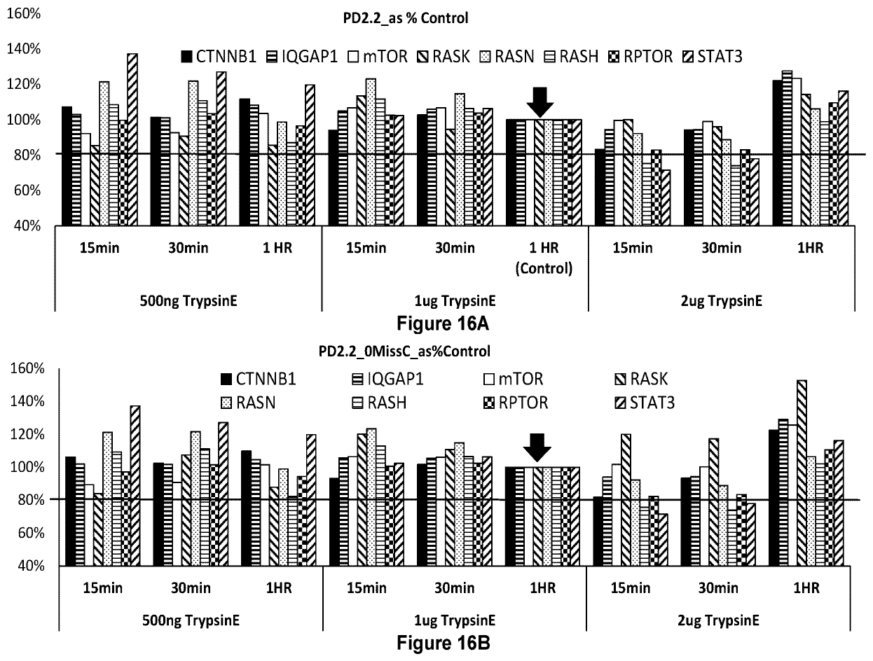

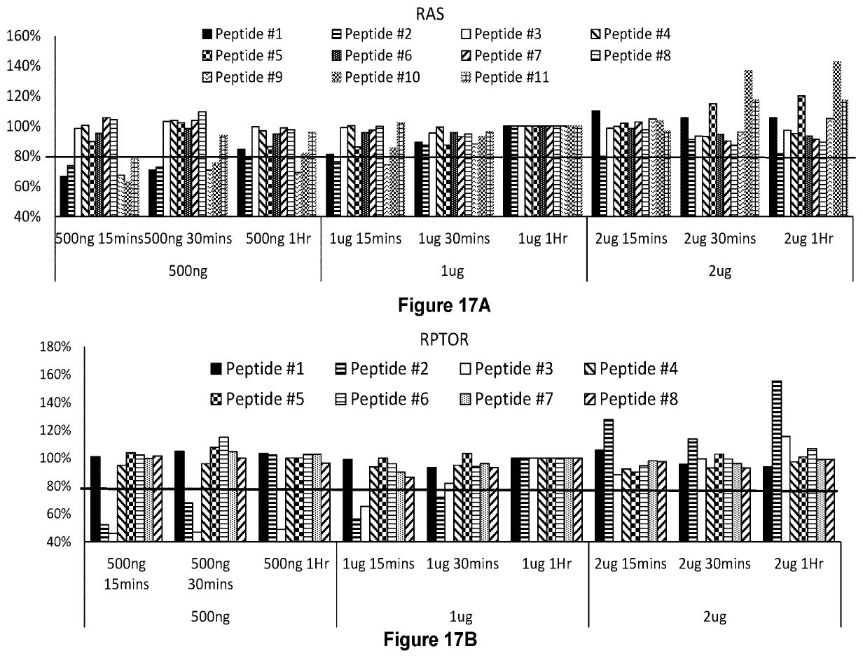

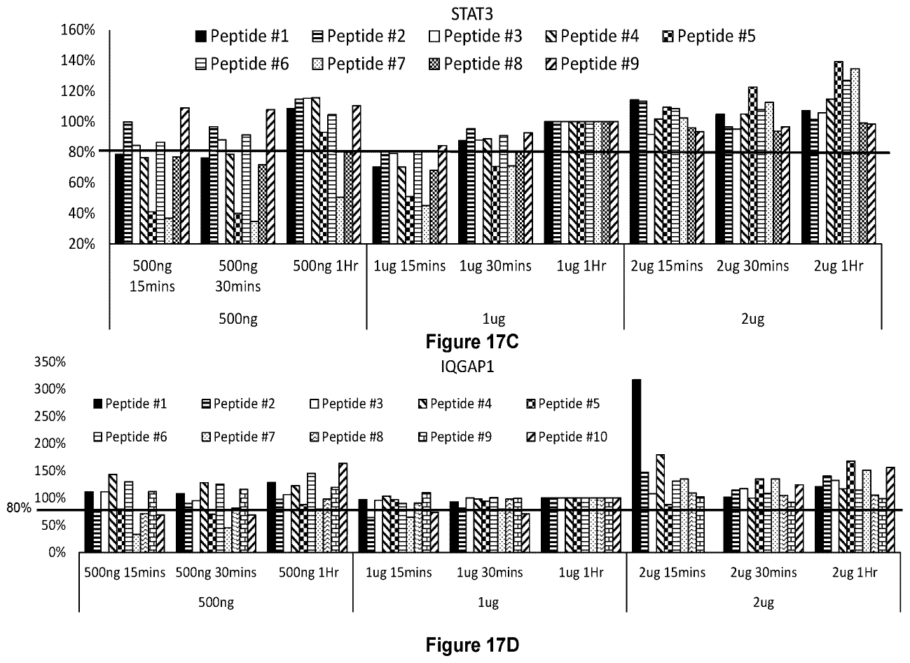

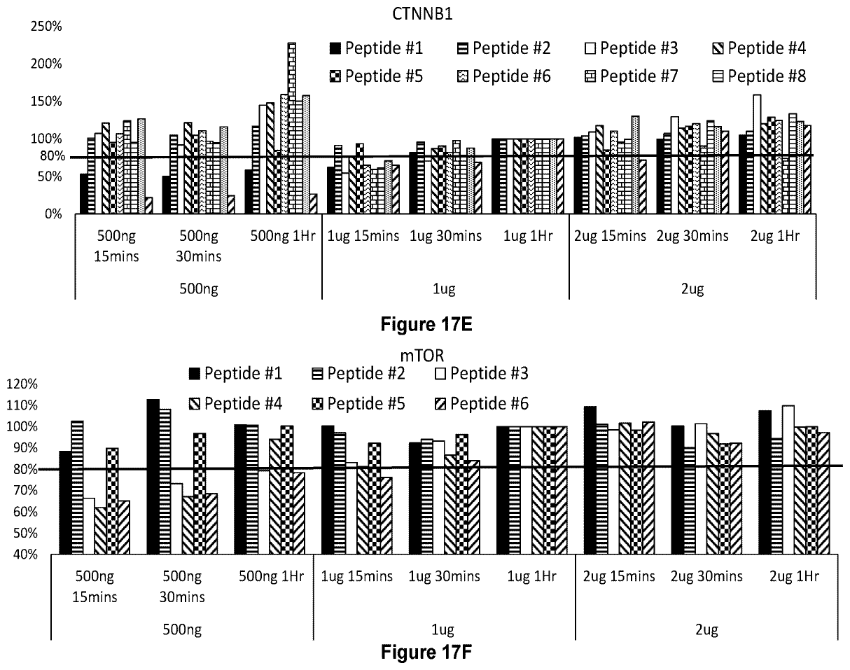

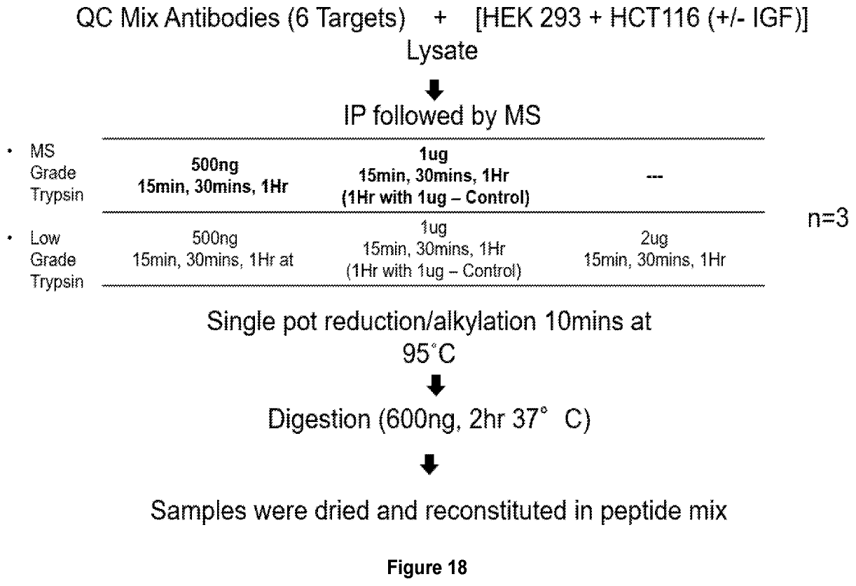

Results are shown in FIGS. 16A-C, 17A-F. All graphs are plotted as % Control (trypsin elution using 1 ug of trypsin for 1 hour) i.e. Peptide Peak Area Intensities (FIG. 16) or PRM ratios (FIG. 17) obtained for all the samples were plotted considering results obtained for samples eluted using 1 μg trypsin for 1 hour as 100.

Elution with 1 ug and 500 ng passes for all times while 2 ug fails for 15 and 30 mins for most of the targets.

MS grade trypsin showed better recovery of targets and low trypsin autolysis peaks in LC-MS analysis.

### Example 9—Optimization of Trypsin Elution from Bead-Time, Amount and Grade of Enzyme

Experiments were performed designed to assess enzymatic elution from IP beads by varying time, trypsin amount, and trypsin grade.

Step 1. Materials used are same as listed in Table 1 with Low grade trypsin. Prepared biotinylated antibody mixture as shown in Table 2 above and mixed with 0.5 mg each of HCT116 (IGF Stim: Unstim)/HEK293 lysates. Performed total of 63 IP reactions by adding appropriate amount of antibody mix in 1.5 mL low protein binding tube. Parafilmed each tube and incubated overnight at 4° C. mixing on a rotator. After incubation added streptavidin magnetic beads using ratio of 1:5 (Antibody: Beads, Since 10 μg Antibody was used 50 μL beads were added per 1 mL). Washed beads with 2× volume cold IP Lysis Buffer twice. Removed wash buffer then added back original volume of IP Lysis Buffer to each tube. Pooled all IPs together in a 50 mL conical tube and aliquoted into 25 1 mL tubes and then added washed beads to each IP. Mixed antibody/antigen samples and rotated for 1 hour at room temperature. Washed 3 times with 500 μL Wash Buffer A. Washed 2 times with 500 μL Wash Buffer B.

Step 2. The following MS sample prep solutions were prepared: 50 mM TEAB—Diluted 1M Trimethylammonium Bicarbonate (TEAB) (PN #90114) to 50 mM by adding 0.5 mL TEAB in 9.5 mL MS grade water, pH 8.5; Single Pot Reduction Alkylation—Final 50 mM TEAB pH 8.5; 10 mM TCEP; 20 mM chloroacetamide (CLAA); 0.2 μg/μL Trypsin Stock—Dissolved 20 μg Trypsin protease (MS grade PN #90057; Low grade PN #1879820) in 100 μL 0.1% Acetic acid; For both types of trypsin-100 ng=Added 0.5 μl 0.2 μg/μl solution+99.5 μl 50 mM TEAB per samples 1-9 MS grade and 28-36 low grade trypsin; 500 ng=Added 2.5 μl 0.2 μg/μl solution+97.5 μl 50 mM TEAB per samples 10-18 MS grade and 37-45 low grade trypsin; 1 μg=Added 5 μl 0.2 μg/μl solution+95 μl 50 mM TEAB per samples 19-27 MS grade and 46-54 low grade trypsin; 1 μg=Added 10 μl 0.2 μg/μl solution+90 μl 50 mM TEAB per samples 55-63 low grade trypsin; trypsin digestion 60 ng/μL=90 μL trypsin stock+60 μL 50 mM TEAB solution then add 10 μL to all samples; 0.2% Formic Acid (FA), 4% Acetonitrile (ACN)—Add 940 μL MS grade water, 20 μL 10% FA and 40 μL of 100% ACN.

Step 3. Mass Spec Sample Prep Various Methods tested included. Samples 1-3=15 min trypsin digestion at 37° C., 100 ng MS grade trypsin; samples 4-6=30 min trypsin digestion at 37° C., 100 ng MS grade trypsin; samples 7-9=1-hour trypsin digestion at 37° C., 100 ng MS grade trypsin; samples 10-12=15 min trypsin digestion at 37° C., 500 ng MS grade trypsin; samples 13-15=30 min trypsin digestion at 37° C., 500 ng MS grade trypsin; samples 16-18=1-hour trypsin digestion at 37° C., 500 ng MS grade trypsin; samples 19-21=15 min trypsin digestion at 37° C., 1 μg MS grade trypsin; samples 22-24=30 min trypsin digestion at 37° C., 1 μg MS grade trypsin; samples 25-27=1-hour trypsin digestion at 37° C., 1 μg MS grade trypsin; samples 28-30=15 min trypsin digestion at 37° C., 100 ng low grade trypsin; samples 31-33=30 min trypsin digestion at 37° C., 100 ng low grade trypsin; samples 34-36=1-hour trypsin digestion at 37° C., 100 ng low grade trypsin; samples 37-39=15 min trypsin digestion at 37° C., 500 ng low grade trypsin; samples 40-42=30 min trypsin digestion at 37° C., 500 ng low grade trypsin; samples 43-45=1-hour trypsin digestion at 37° C., 500 ng low grade trypsin; samples 46-48=15 min trypsin digestion at 37° C., 1 μg low grade trypsin; samples 49-51=30 min trypsin digestion at 37° C., 1 μg low grade trypsin; samples 52-54=1-hour trypsin digestion at 37° C., 1 μg low grade trypsin; samples 55-57=15 min trypsin digestion at 37° C., 2 μg low grade trypsin; samples 58-60=30 min trypsin digestion at 37° C., 2 μg low grade trypsin; samples 61-63=1-hour trypsin digestion at 37° C., 2 μg low grade trypsin;

Step 4. The sample prep continued for trypsin elution by adding stated amounts and type of trypsin in 100 μl 50 mM TEAB to each sample and incubated for stated times at 37° C. in a thermomixer shaking at 500 rpm.

Step 5. After 1 HR incubation tubes were placed onto magnet to remove beads and 90 μL of supernatant was removed and 2 μL 25 ng/μL GFP and 25 μl single pot reduction/alkylation solution were added to each reaction and incubated at 95° C. for 5 mins.

Step 6. After Reduction/Alkylation added 60 ng/μl digestion trypsin was added and incubated at 37° C. for 2 hours shaking at 500 rpm.

Step 7. Acidified the samples by adding 2.5 μL 25% TFA to acidify samples (pH <3). Centrifuged all samples at 15,000×g for 2 mins and then removed 112 μL of samples and transferred to clean 1.5 ml low protein binding tube. Dried down the samples for about 1 hr in speed vac. Added 17 μL of QC peptide Mix (80 fmol) in 0.2% FA and 4% ACN to samples 1-18. Reconstitute samples 19-24 in 17 μL 4% ACN and MS grade water. Stored all samples at −20° C. before nanoLC-MS/MS analysis.

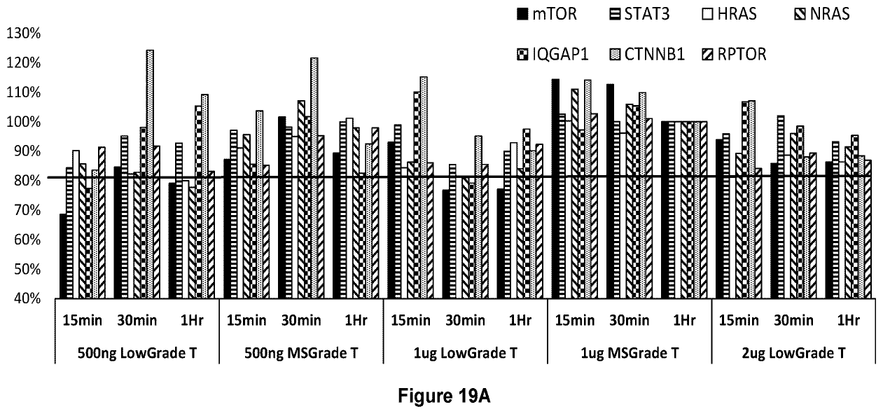

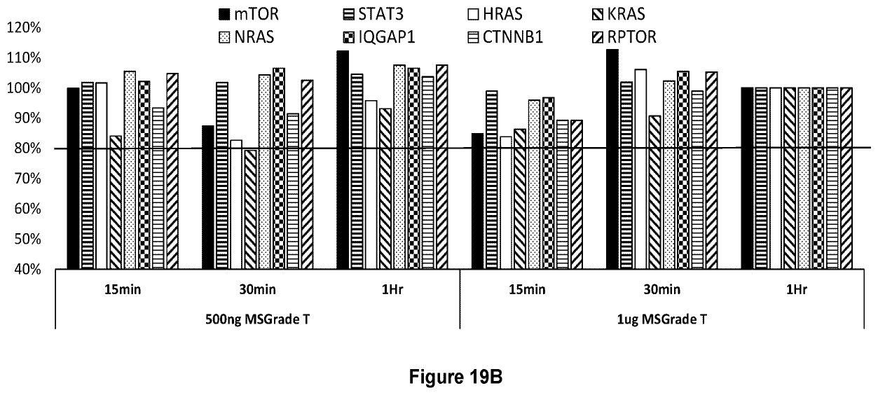

Results are shown in FIGS. 19A-B. Graphs are plotted as % Control (Samples eluted using 1 ug MS grade trypsin for 1 hour).

Elution using 500 ng Low grade or MS grade trypsin at all time points showed drop in intensities for most of the targets.

1 ug Low grade—drop in intensities, 2 failed at 30 mins, 1 at 1 hour.

1 ug MS grade—equivalent or better intensities for 15 and 30 mins as compared to elution at 1 hour.

2 ug Low grade—all targets pass, slightly lower intensities as compared to control.

KRAS with low grade trypsin showed 400-500% increase compared to control.

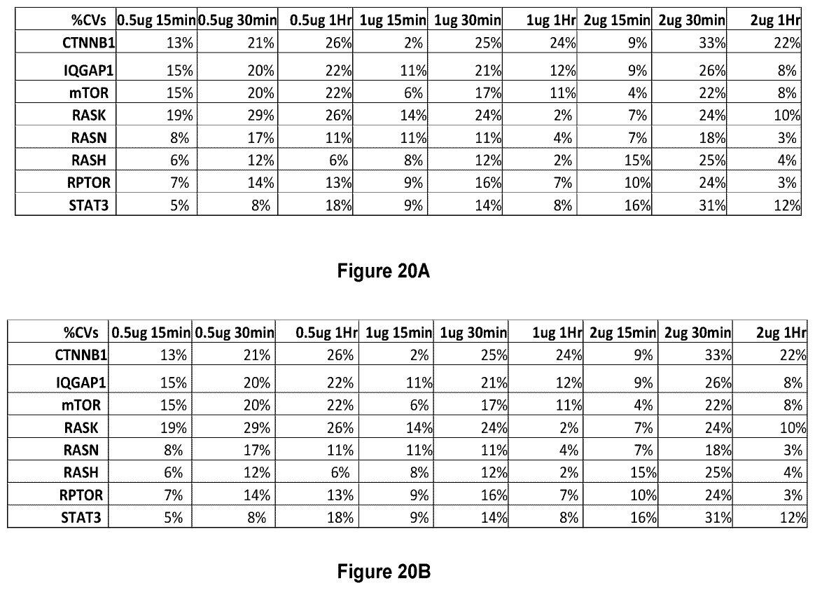

FIG. 20A-B provides tables showing % CVs for Example 8 above (FIG. 20A) and this Example 9 (FIG. 20B).

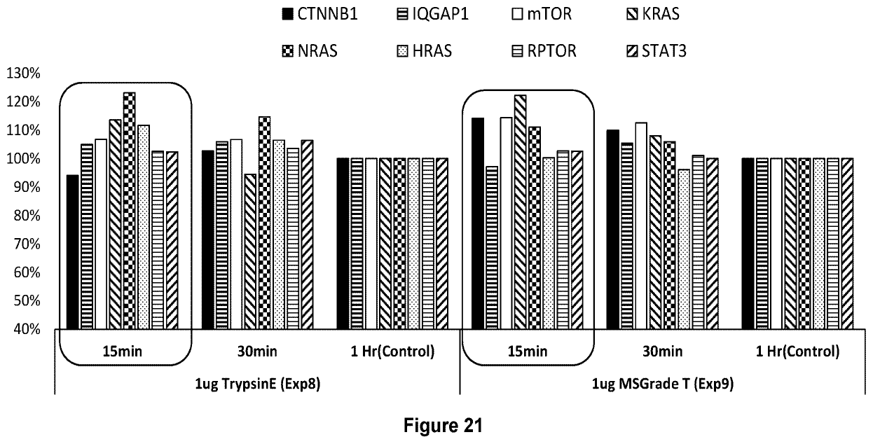

FIG. 21 provides a comparison of results for 1 μg trypsin elution from Examples 8 and 9.

### Example 10: Certain Embodiments

The following numbered items provide additional support for and descriptions of the embodiments herein.

Item 1. A method for detecting one or more target protein(s) in a biological sample, comprising
a. enriching the target protein(s) from a biological sample by binding the target protein(s) to a solid support;
b. fragmenting the enriched target protein(s) by:
   i. while bound to the solid support, treating the enriched target protein(s) with a first enzymatic digestion,
   ii. reducing and alkylating the digested target protein(s) in a single reaction vessel, and
   iii. digesting the reduced, alkylated, and digested target protein(s) in a second enzymatic digestion, wherein the second enzymatic digestion is allowed to proceed for up to 4 to 18 hours; and
c. detecting one or more target protein(s) in the sample.

Item 2. The method of item 1, wherein the solid support comprises a bead or a resin.

Item 3. The method of item 1, wherein the solid support comprises a magnetic bead.

Item 4. The method of item 1, wherein the solid support comprises an immunoaffinity bead.

Item 5. The method of item 1, wherein enriching the target protein(s) from a biological sample by binding the target protein(s) to a solid support comprises treating the biological sample with at least one antibody capable of immunoaffinity enriching the target protein(s) from a biological sample.

Item 6. The method of any one of items 1-5, wherein detecting one or more target proteins(s) in the sample comprises assaying the fragmented protein(s) via mass spectrometry to determine the presence or absence of at least one peptide from the target protein(s).

Item 7. The method of item 6, wherein the peptide is less than or equal to 40 amino acids in length.

Item 8. The method of any one of items 1-7, wherein detecting one or more target protein(s) in the sample comprises ELISA, Western blot, Luminex, fluorescence-based imaging, or chemiluminescent-based imaging.

Item 9. The method of any one of items 1-8, wherein the first and/or second enzymatic digestion comprises digestion with trypsin, chymotrypsin, AspN, GluC, LysC, LysN, ArgC, proteinase K, pepsin, clostripain, elastase, GluC biocarb, LysC/P, LysN promisc, protein endopeptidase, staph protease or thermolysin.

Item 10. The method of item 9, wherein the first and/or second enzymatic digestion comprises digestion with trypsin.

Item 11. The method of item 9, wherein the first and/or second enzymatic digestion comprises digestion with trypsin and LysC.

Item 12. The method of any one of items 9-11, wherein the trypsin is present in the first enzymatic digestion at a concentration of 0.1 μg/μl to 0.4 μg/μl.

Item 13. The method of any one of items 9-11, wherein the trypsin is present in the second enzymatic digestion at a concentration of 0.02 μg/μl to 0.08 μg/μl.

Item 14. The method of any one of items 1-13, wherein the reduction/alkylation step comprises mixing the product of the first enzymatic digestion with a solution comprising TCEP and chloroacetamide.

Item 15. The method of item 14, wherein the TCEP and chloroacetamide are present in a ratio of 1:1, 1:2, 1:3, 1:4, or 1:5.

Item 16. The method of items any one of 1-15, further comprising the step of neutralization after the second digestion and prior to mass spectrometry.

Item 17. The method of item 16, wherein the neutralization step comprises adding trifluoroacetic acid (TFA) to the product of the second enzymatic digestion.

Item 18. The method of any one of items 1-17, wherein step a) comprises treating the sample with a labelled antibody capable of binding to the target protein to provide a labelled antibody-protein conjugate; and binding the labelled antibody-protein conjugate with a capture agent capable of binding to the labelled antibody to isolate the target protein from the sample.

Item 19. The method of item 17, wherein the label is biotin and the capture agent is streptavidin.

Item 20. The method of items any one of 1-19, wherein the lower limit of detection for the protein(s) is from 0.04 to 11.11 fmol.

Item 21. The method of any one of items 1-20, further comprising determining the quantity of the target protein.

Item 22. The method of item 21, wherein the quantity of a target protein is determined by adding an internal standard peptide of known amount to the digested protein prior to mass spectrometry, wherein the internal standard peptide has the same amino acid sequence as a target peptide, and is detectably labeled, and determining the quantity of a target peptide by comparison to the internal standard.

Item 23. The method of item 21, wherein the quantity of a target protein is determined by a method comprising comparing an amount of a target peptide in the sample to the amount of the same target peptide in a control sample.

Item 24. The method of any one of items 21 to 23, further comprising quantifying the relative amount of the target protein.

Item 25. The method of any one of items 21 to 24, further comprising quantifying the absolute amount of the target protein.

Item 26. The method of any one of items 21 to 25, wherein the lower limit of quantification is from 0.04 to 11.11 fmol.

Item 27. The method of any one of items 1-26, further comprising desalting after fragmentation and prior to mass spectrometry.

Item 28. The method of any one of items 1-27, wherein the mass spectrometry is selected from targeted mass spectrometry and discovery mass spectrometry.

Item 29. The method of item 21, wherein the targeted mass spectrometry is selected from multiple reaction monitoring (MRM), selected reaction monitoring (SRM), and parallel reaction monitoring (PRM), or combinations thereof.

Item 30. The method of any one of items 1-29, wherein the biological sample is selected from isolated cells, plasma, serum, whole blood, CSF, urine, sputum, tissue, and tumorous tissue.

Item 31. The method of any one of items 1-30, wherein the biological sample is human.

Item 32. The method of any one of items 1-31, wherein the peptide from the target protein(s) comprises an epitope for the antibody capable of immunoaffinity enriching the target protein(s).

Item 33. The method of any one of items 1-14, wherein the digestion is complete in 4 hours or less.

Item 34. The method of any one of items 1-5, wherein the method further comprises separating the solid support from digested protein(s).

We claim:

1. A method for detecting one or more target protein(s) in a biological sample, comprising a. enriching the target protein(s) from a biological sample by binding the target protein(s) to a solid support;
b. fragmenting the enriched target protein(s) by:
i. while bound to the solid support, treating the enriched target protein(s) with a first enzymatic digestion,
ii. reducing and alkylating the digested target protein(s) in a single reaction vessel, and
iii. digesting the reduced, alkylated, and digested target protein(s) in a second enzymatic digestion, wherein optionally the second enzymatic digestion is allowed to proceed for up to 18 hours;
c. detecting one or more target protein(s) in the sample.

2. The method of claim 1, wherein enriching the target protein(s) from a biological sample by binding the target protein(s) to a solid support comprises treating the biological sample with at least one antibody capable of immunoaffinity enriching the target protein(s) from a biological sample.

3. The method of claim 1, wherein detecting one or more target proteins(s) in the sample comprises assaying the fragmented protein(s) via mass spectrometry to determine the presence or absence of at least one peptide from the target protein(s).

4. The method of claim 3, wherein the peptide is less than or equal to 40 amino acids in length.

5. The method of claim 1, wherein detecting one or more target protein(s) in the sample comprises ELISA, Western blot, bead-based multianalyte profiling, fluorescence-based imaging, or chemiluminescent-based imaging.

6. The method of claim 1, wherein the first and/or second enzymatic digestion comprises digestion with trypsin, chymotrypsin, AspN, GluC, LysC, LysN, ArgC, proteinase K, pepsin, clostripain, elastase, GluC biocarb, LysC/P, LysN promise, protein endopeptidase, staph protease or thermolysin.

7. The method of claim 1, wherein the first and/or second enzymatic digestion comprises digestion with trypsin.

8. The method of claim 1, wherein the first and/or second enzymatic digestion comprises digestion with trypsin and LysC.

9. The method of claim 1, wherein the reduction/alkylation step comprises mixing the product of the first enzymatic digestion with a solution comprising TCEP and chloroacetamide.

10. The method of claim 9, wherein the TCEP and chloroacetamide are present in a ratio of 1:1, 1:2, 1:3, 1:4, or 1:5.

11. The method of claim 1, further comprising the step of neutralization after the second digestion and prior to mass spectrometry.

12. The method of claim 11, wherein the neutralization step comprises adding trifluoroacetic acid (TFA) to the product of the second enzymatic digestion.

13. The method of claim 1, wherein step a) comprises treating the sample with a labelled antibody capable of binding to the target protein to provide a labelled antibody-protein conjugate; and binding the labelled antibody-protein conjugate with a capture agent capable of binding to the labelled antibody to isolate the target protein from the sample.

14. The method of claim 13, wherein the label is biotin and the capture agent is streptavidin.

15. The method of claim 1, wherein the lower limit of detection for the protein(s) is from 0.04 to 11.11 fmol.

16. The method of claim 1, further comprising determining the quantity of the target protein by adding an internal standard peptide of known amount to the digested protein prior to mass spectrometry, wherein the internal standard peptide has the same amino acid sequence as a target peptide, and is detectably labeled, and determining the quantity of a target peptide by comparison to the internal standard.

17. The method of claim 16, wherein the quantity of a target protein is determined by a method comprising comparing an amount of a target peptide in the sample to the amount of the same target peptide in a control sample.

18. The method of claim 1, wherein the peptide from the target protein(s) comprises an epitope for the antibody capable of immunoaffinity enriching the target protein(s).

19. The method of claim 1, wherein the digestion is complete in 4 hours or less.

20. The method of claim 1, wherein the method further comprises separating the solid support from digested protein(s).
